SEQUENCE LISTING (7 sequences)

SEQ ID NO: 1 - protein, 497 aa, Homo sapiens [sequence omitted]

SEQ ID NO: 2 - protein, 497 aa, Homo sapiens
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPANATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 3 - protein, 497 aa, Homo sapiens
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLLNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPANATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 4 - protein, 497 aa, Homo sapiens
ARPCNPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 5 - protein, 497 aa, Homo sapiens
ARPCIPKSFGYSSVVCVCNATYCDSFDPPTYPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 6 - protein, 497 aa, Homo sapiens
ARPCIPKSFGYSSVVCVCNATYCDSFDPPTFPAQGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 7 - protein, 497 aa, Homo sapiens
ARPCIPKSFGYSSVVCVCNATYCDSFDPPTFPALGTFSRYESTRSGRRMELSTGTIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

(19) United States
(12) Patent Application Publication
(10) Pub. No.: US 2022/0325265 A1
(43) Pub. Date: Oct. 13, 2022

(54) GLUCOCEREBROSIDASE MUTANTS

(71) Applicant: Hoffmann-la Roche Inc., Little Falls, NJ (US)

(72) Inventors: Joerg BENZ, Rheinfelden (DE); Martin EBELING, Grenzach-Wyhlen (DE); Ravi JAGASIA, Basel (DE); Nina Marion PFAHLER, Basel (DE); Ralf THOMA, Basel (DE)

(73) Assignee: Hoffmann-La Roche Inc., Little Falls, NJ (US)

(21) Appl. No.: 17/641,740

(22) PCT Filed: Sep. 7, 2020
(86) PCT No.: PCT/EP2020/074888

§ 371 Date: Mar. 9, 2022

(30) Foreign Application Priority Data
Sep. 9, 2019 (EP) 19196078.8

(51) Int. Cl. C12N 9/24; A61K 38/47; A61K 47/64; A61P 25/28

(52) U.S. Cl. CPC C12N 9/2402; C12Y 302/01045; A61K 38/47; A61K 47/64; A61P 25/28

(57) ABSTRACT

The present invention provides variants of human β-Glucocerebrosidase protein.

The enzyme glucocerebrosidase (EC 3.2.1.45), also called acid β-glucosidase/0-glucosylceramidase or short GCase/GBA1 is classified into the enzymatic class of hydrolases, indicating the ability of hydrolyzing naturally occurring glucosphingolipids including glucosylceramide (GlcCer; also known as glucocerebroside) or glucosylsphingosine (GlcSph) into glucose and ceramide/sphingosine, respectively (Brady et al., 1965). Glucocerebrosidase is a lysosomal luminal membrane-associated hydrolase, where it interacts with glucolipids. Inherited autosomal, recessive mutations of the related glucosylceramidase beta (GBA) gene lead to deficiency in activity and the clinical manifestation of Gaucher disease (GD) (Beutler and Grabowski, 1994). Since the discovery of GBA mutations causing GD, 300 different mutations have been identified in GD patients with a prevalence of 1 in 50000 people in the general population (Beutler et al., 2005; Hruska et al., 2008; Charrow et al., 2000; Horowitz et al., 1998).

Glucocerebrosidase is a lysosomal enzyme, responsible for the degradation of glucosylsphingolipids. When dysfunctional, the accumulation of substrate induces an altered inflammatory response in tissue macrophages (Gaucher cells) and other related cells (Kornhaber et al., 2008; Schetz & Shankar, 2004; Smith et al., 2017). Gaucher cells are the most prominent pathological hallmark together with mononuclear phagocytes and neuronal cells in the brain, which are involved in the pathology of GD (Beutler & Grabowski, 2001). Philippe Gaucher first described the clinical properties of this disease in 1882 (Gaucher, 1882), which is nowadays reported as the most common lysosomal storage disorder (Grabowski, 2008). Symptoms of this disease are inter alia anemia, enlargement of liver and spleen, bone lesions and in severe cases neurologic manifestations, widely classified into three clinical subtypes (Beutler et al., 1994; Kornhaber et al., 2008). The most common form is classified into type I Gaucher disease with nearly no neuronopathic implications, currently treatable with the use of enzyme replacement therapy (ERT). Type II and III are more severe types of GD with type II having acute neuronopathic phenotype, indicating symptoms at near birth, progressing until death during early infancy. Type III causes chronic severe neuropathic symptoms, including learning disabilities, cardiac abnormalities and myolonic epilepsy (Beutler et al., 1994; Sidransky et al., 2007; Sidransky & Lopez, 2012).

300 mutations have been identified and directly linked to the progression of GD, resulting in a wide pathological spectrum of this disorder (Alfonso et al., 2007; Grabowski & Horowitz, 1997). Inherited missense and nonsense mutations in the GBA gene can lead to misfolding, mistrafficking and destabilization of the glucocerebrosidase enzyme. Two of the most frequent mutations found in the literature are the mutations N370S and L444P, accounting for 50% of all known GBA mutations. Patients carrying the mutated GCase variant N370S are usually diagnosed with type I GD, possessing a wide range of symptoms. N370S is responsible for conserving and stabilizing proper conformation of the active binding pocket (Lieberman et al., 2007). L444P reveals a more severe type categorized in type II or III when carrying this GBA gene mutation, possibly disrupting the hydrophobic structure, altering the domain II function (Lieberman et al., 2007). However, since there is a lot of genotype to phenotype variability, the severity of the mutation on GD cannot be directly correlated yet to different structural changes. Interestingly, heterozygous mutations N370S and more severe L444P are frequently found within Parkinson disease (PD) patients, with L444P GD patients studied being at higher risk developing PD (Sidransky et al., 2009). It still remains unclear though to which extend Gaucher disease patients are likely to develop Parkinsonsim and what the link between these two disorders constitutes (Tayebi et al., 2003).

Until today, the most effective treatment approach for GD is the enzyme replacement therapy (ERT) with Cerezyme® (imiglucerase, Genzyme Corp.™). The molecule was approved in 1994 and successfully treats peripheral symptoms of type I GD patients (Grabowski, 2008; Van Rossum & Holsopple, 2016). Ceredase™ (Genzyme Corp.™), an alglucerase targeting macrophages has been approved in 1991, highly reducing the sphingosine levels within Gaucher cells (Barton et al., 1990, 1991; Sato & Beutler, 1993). Both released products are mainly treating GD type I patients by preventing progressive manifestation in the periphery. Other than that, VPRIV® (velaglucerase, approved 2010) produced in human fibroblasts by Shire (Zimran et al., 2010) and ELELYSO® (taliglucerase alfa, approved 2012) produced in carrot cells by Pfier and Protalix (Abrahamov et al., 2016) are two newer developed ERTs for type I GD patients.

With enzyme enrichment therapy (EET), GCase deficiency in patients should be restored by e.g. small molecular chaperones helping to fold and stabilize the protein as well as proper trafficking within target cells and lysosomes. Another considered approach is the substrate reduction therapy (SRT), where substrate levels should be reduced by slowing down or disrupting the glucolipid biosynthesis (Grabowski, 2008; Vunnam & Radin, 1980). Here, ZAVESCA® (Miglustat, approved 2002, Oxford GlycoSciences/Actelion) and Eliglustat (Cerdelga®, Sanofi-Genzyme, approved 2014) are two approved oral inhibitors of glucosylceramide synthase, reversibly competing with its natural substrates used for type I GD therapy (Butters et al., 2000).

One interesting avenue of investigation is to search for more stable GCase molecules with enhanced functionality that can be more efficacious in vitro to in vivo. Several stabilized mutants were already reported as the hGCase mutants K321N and K321N/H145L of Amicus Therapeutics (herein Amicus) (Hung, 2015; U.S. Pat. No. 8,962,564). Both mutations were introduced on top of the R495H_{CHO} sequence, which carries the mutation in position 495 (R->H). The single mutant K321N carries an additional modification replacing lysine (K) to asparagine (N) in position 321 within the α6-helix based on sequence alignment with bull GCase (B. taurus). The double mutant K321N/H145L harbors an additional substitution on top of the K321N sequence with histidine (H) replaced by leucine (L) in position 145, indicating a modification between the β-sheet and the α2-helix based on sequence alignments with B. taurus (bull) and S. scrofa (pig) (Hung, 2015).

The aim of the present invention was to provide GCase variants with enhanced functionality that can be more efficacious in vitro to in vivo.

The present invention provides a recombinant human β-Glucocerebrosidase protein, wherein the protein comprises at least one substitution relative to Seq. Id. No. 1, and wherein the substitution is selected from the group consisting of: I5N, F31Y, L34Q, M53T and P55T, and combinations thereof.

In a particular embodiment the recombinant human β-Glucocerebrosidase protein comprises at least two substitutions relative to Seq. Id. No. 1, wherein the two substitutions are M53T and P55T.

In a particular embodiment the recombinant human β-Glucocerebrosidase protein comprises an amino acid sequence selected from the group consisting of Seq. Id. No. 4-7.

In a particular embodiment the recombinant human β-Glucocerebrosidase protein comprises an amino acid sequence set forth in Seq. Id. No. 7.

In a second aspect the present invention provides an isolated nucleic acid encoding the recombinant human β-Glucocerebrosidase protein of the present invention.

In a third aspect the present invention provides a vector comprising the nucleic acid sequence of the present invention.

In a fourth aspect the present invention provides a host cell comprising the vector of the present invention.

In a further aspect the present invention provides a pharmaceutical formulation comprising the recombinant human β-Glucocerebrosidase protein of the present invention.

In a further aspect the present invention relates to a recombinant human β-Glucocerebrosidase protein of the present invention for use as a medicament.

In a particular embodiment the present invention relates to a recombinant human β-Glucocerebrosidase protein of the present invention for use in the treatment of a neurodegenerative disease, in particular Gaucher's disease and Parkinson's disease.

In a further aspect the present invention provides a conjugate comprising a human β-Glucocerebrosidase protein of present invention and a blood brain barrier shuttle and the use of such a conjugate for the treatment of a neurodegenerative disease, in particular Gaucher's disease and Parkinson's disease.

### SHORT DESCRIPTION OF THE FIGURES

FIG. 1: Raw data plot (upper section of graph) and first derivative peak (bottom section) of the nanoDSF measurement. (A) Raw data and first derivative graph of the references R495H_{S2}, K321N, K321N/H145L and R495H_{CHO}. (B) Raw data plot and first derivative graph of I5N, F31Y, L34Q and M53T/P55T. Sample concentration: 0.06-0.5 mg/ml, temperature gradient: 3° C./min, excitation power: 20-80%.

FIG. 2: Residual GCase activities measured after incubation of hGCase mutants at 55° C. in the waterbath. Dark grey bars: 5 min incubation time; light grey bars: 10 min incubation time. Residual activity calculated as percentage of initial GCase activity (100%; before heating). Protein concentration used: 50 nM. Horizontal line represents residual activity of R495H.

FIG. 3: pH profiles evaluated for hGCase references R495H_{CHO} (A), R495H_{S2} (B), Amicus single mutant K321N (C) and variant M53T/P55T (D). Buffers tested: 20 mM citrate buffer (pH 3.5-5), 20 mM MES buffer (pH 6-6.5), 20 mM HEPES buffer (pH 7-8). Highest activity of each data set was normalized to 100%.

FIG. 4: (A) and (B) IC50 determination with reversible inhibitor IFG. (C) and (D) IC50 determination with irreversible inhibitor CBE. (A,C) IC50 of hGCase mutant M53T/P55T, (B,D) IC50 of all tested variants. IFG concentration range: 0.02-100 μM; CBE concentration range: 0.04-2500 μM. Protein concentration: 25 nM, respectively; Pre-incubation of enzyme with inhibitor for 10 min at RT before activity measurement.

FIG. 5: Determination of the kinetic parameters KM and Vmax for representative mutant M53T/P55T. (A) Raw signal data (progression curves) in RFU measured with increasing incubation time. (B) Michaelis-Menten fit. Measurements done using activity assay described in section 2.3.5. Res-β-glc concentration range: 4-500 μM. Curve fit in (B) and final calculations via GraphPad Prism7 (Michealis-Menten nonlinear fit following equation 4). ¹ initially measured GCase activity (25 mM) in RFU. 2 activation after adding phosphatidylserine (4 μM).

FIG. 6: Initial activity (no added lipid) of the tested hGCase mutants (light grey bars) plotted against the activation with added phosphatidylserine (dark grey bars). Phosphatidylserine concentration 4 μM, protein concentration: 25 nM. Pre-incubation of lipid+enzyme: 10 min at RT.). ¹ initially measured GCase activity (25 mM) in RFU. 2 activation after adding phosphatidylserine (4 μM).

FIG. 7: Phosphatidylserine activation of hGCase mutants expressed in fold increase of initial activity. Phosphatidylserine concentration: 4 μM, protein concentration: 25 nM. Pre-incubation of lipid+enzyme: 10 min at RT. Horizontal line represents mean R495H activation level.

FIG. 8: Activity levels normalized to hGCase protein levels measured after treatment of H4 glioblastoma GBA-KO cells with hGCase molecules. Treatment concentration (hGCase protein): 1, 10 and 100 nM (100 nM not shown herein), respectively. Activity and protein amount measurement after 2 h of incubation. Activity measured with artificial substrate res-β-glc (20 μM); protein concentration determination via AlphaLISA (section 2.3.6). H4 wt cells (GCase levels normalized to 100%) and H4 GBA-KO cells (loss of GBA gene) served as control. ¹ GCase activity measured in H4 cells normalized to activity measured in H4 (wt) cells designated to 100% and normalized to protein amount. ² H4 glioblastoma cells with knock-out GBA gene.

FIG. 9: Reduction of the natural substrate GlcSph after treatment of H4 glioblastoma GBA-KO cells with hGCase molecules. Treatment concentration (hGCase protein): 1, 10 and 100 nM, respectively. Analysis of substrate levels after 48 h of incubation via LC/MS. H4 wt cells (GCase levels normalized to 100%) and H4 GBA-KO cells (loss of GBA gene) served as control. ¹ measured glucosylsphingosine (GlcSph) levels normalized to total protein amount. ² H4 glioblastoma cells with knock-out GBA gene.

FIG. 10: Reduction of the natural substrate GlcSph after treatment of H4 glioblastoma GBA-KO cells with hGCase molecules. Regraphed data of FIG. 9 with results of 1 and 10 nM protein treatment on GBA-KO H4 cells in direct comparison. ¹ measured glucosylsphingosine (GlcSph) levels normalized to total protein amount. ² n.s=not significant; ³ H4 glioblastoma cells with knock-out GBA gene.

FIG. 11: the compiled spiderweb charts are presented for each individual GCase mutant to draw a final performance profile. 11A: GCase Mutant I5N; 11B: GCase Mutant F31Y; 1 IC: GCase Mutant K321N, K321N/H145L; 11A: GCase Mutant L34Q; 11A: GCase Mutant M53T+P55T;

### DETAILED DESCRIPTION OF THE INVENTION

Unless noted differently, variant, recombinant human β-glucocerebrosidase protein properties are stated relative to recombinant human β-glucocerebrosidase protein having the amino acid sequence set forth in Seq. Id. No. 1. The β-glucocerebrosidase protein having the amino acid sequence set forth in Seq. Id. No. 1 carries the mutation R to H in position 495 relative to wildtype β-glucocerebrosidase protein i.e. arginin at position 495 is replaced by histidin. Herein, “GCase” is an abbreviation used for 3-glucocerebrosidase. All amino acid numbers are relative to Seq. Id. No. 1. Thus, position 145 would be the 145th amino acid occurring in Seq. Id. No. 1. Furthermore, variant, recombinant f-glucocerebrosidase proteins as disclosed herein also include functional fragments or derivatives thereof.

The “blood-brain barrier” or “BBB” refers to the physiological barrier between the peripheral circulation and the brain and spinal cord (i.e., the CNS) which is formed by tight junctions within the brain capillary endothelial plasma membranes, creating a tight barrier that restricts the transport of molecules into the brain, even very small molecules such as urea (60 Daltons). The blood-brain barrier within the brain, the blood-spinal cord barrier within the spinal cord, and the blood-retinal barrier within the retina are contiguous capillary barriers within the CNS, and are herein collectively referred to a the blood-brain barrier or BBB. The BBB also encompasses the blood-CSF barrier (choroid plexus) where the barrier is comprised of ependymal cells rather than capillary endothelial cells.

The term “blood brain barrier shuttle” as used herein refers to a molecule which enables transport of a cargo molecule coupled/linked to the blood brain barrier shuttle across the BBB. Suitable blood brain barrier shuttles are for example antibodies or antibody fragments binding to a receptor expressed on the BBB such as e.g. transferrin receptor. Exemplary blood brain barrier shuttles are anti-transferrin receptor antibodies such as e.g. disclosed in WO 2014/033074 and WO 2012/075037. In a particular embodiment the blood brain barrier shuttle is a Fab antibody fragment or a scFab antibody fragment directed to the human transferrin receptor, preferably a cross Fab antibody fragment. Exemplary cross Fab fragments are described in WO 2009/080251, WO 2009/080252 and MABS 2016, VOL. 8, NO. 6, 1010-1020.

A “conjugate” is a GCase protein of the present invention conjugated to one or more heterologous molecule(s), including but not limited to a blood brain barrier shuttle.

Conjugation may be performed using a variety of chemical linkers. For example, the GCase protein of the present invention and the blood brain barrier shuttle may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HC1), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). The linker may be a “cleavable linker” facilitating release of the effector entity upon delivery to the brain. For example, an acid-labile linker, peptidase-sensitive linker, photolabile linker, dimethyl linker or disulfide-containing linker (Chari et al, Cancer Res. 52: 127-131 (1992); U.S. Pat. No. 5,208,020) may be used.

Covalent conjugation can either be direct or via a linker. In certain embodiments, direct conjugation is by construction of a protein fusion (i.e., by genetic fusion of the two genes encoding the GCase protein and blood brain barrier shuttle and expressed as a single protein). In certain embodiments, direct conjugation is by formation of a covalent bond between a reactive group on one of the two portions of the GCase protein and a corresponding group or acceptor on the blood brain barrier shuttle. In certain embodiments, direct conjugation is by modification (i.e., genetic modification) of one of the two molecules to be conjugated to include a reactive group (as non-limiting examples, a sulfhydryl group or a carboxyl group) that forms a covalent attachment to the other molecule to be conjugated under appropriate conditions. As one non-limiting example, a molecule (i.e., an amino acid) with a desired reactive group (i.e., a cysteine residue) may be introduced into, e.g., the blood brain barrier shuttle and a disulfide bond formed with the GCase protein. Conjugation may also be performed using a variety of linkers. For example, a GCase protein and a blood brain barrier shuttle may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HC1), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Peptide linkers, comprised of from one to twenty amino acids joined by peptide bonds, may also be used. In certain such embodiments, the amino acids are selected from the twenty naturally-occurring amino acids. In certain other such embodiments, one or more of the amino acids are selected from glycine, alanine, proline, asparagine, glutamine and lysine. The linker may be a “cleavable linker” facilitating release of the effector entity upon delivery to the brain. For example, an acid-labile linker, peptidase-sensitive linker, photolabile linker, dimethyl linker or disulfide-containing linker (Chari et al, Cancer Res. 52: 127-131 (1992); U.S. Pat. No. 5,208,020) may be used.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

In a further aspect, provided are pharmaceutical compositions comprising any of the recombinant human β-Glucocerebrosidase variant provided herein, e.g., for use in any of the below therapeutic methods. In one aspect, a pharmaceutical composition comprises any of the recombinant human β-Glucocerebrosidase variant provided herein and a pharmaceutically acceptable carrier. In another aspect, a pharmaceutical composition comprises any of the recombinant human β-Glucocerebrosidase variant provided herein and at least one additional therapeutic agent.

Pharmaceutical compositions of a recombinant human β-Glucocerebrosidase variant as described herein are prepared by mixing such Gcase variant having the desired degree of purity with one or more optional pharmaceutically acceptable carriers (Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The pharmaceutical composition herein may also contain more than one active ingredients as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Pharmaceutical compositions for sustained-release may be prepared. Suitable examples of sustained-release preparations include semipermeable matrices of solid hydrophobic polymers containing the antibody, which matrices are in the form of shaped articles, e.g., films, or microcapsules.

The pharmaceutical compositions to be used for in vivo administration are generally sterile. Sterility may be readily accomplished, e.g., by filtration through sterile filtration membranes.

Any of the Gcase variant provided herein may be used in therapeutic methods.

In a further aspect, the invention provides a method for treating a neurodegenerative disease, in particular Gaucher's disease and Parkinson's disease. In one aspect, the method comprises administering to an individual having such a neurodegenerative disease an effective amount of a recombinant human β-Glucocerebrosidase variant as described herein. An “individual” according to any of the above aspects may be a human.

Material and Methods

Expression System and Expression Plasmid

The expression vector pExpreS2_1-A was used for all hGCase variants for the gene expression in the S2 cell system (Schneider S2 Drosophila cell line). The expression system is based on an insect cell line of Drosophila melanogaster. The expression was done constitutively, therefore no inducing agent was needed. Zeocin served as selective agent for stable cell line generation (1.5 mg/ml). The basic genotype for the respective plasmids was pExpreS2_1-A-BIP-hGCase (1-497, X, R495H)-GS-Sor-8×His, were X is substituted by each suggested mutation listed in the following table (table 1). Thereby, the His8-tag is coupled to the sequence via a glycine-serine linker (GS), including a sortase recognition site (Sor). The following table provides an overview of the established hGCase with the abbreviations used in this application as well as their biochemical properties.

TABLE 1
hGCase mutants with related biochemical properties.

| | | Molecular | Extinction | |
|---|---|---|---|---|
| | | weight² | coefficient² | Seq. |
| GCase variants | Abbreviation | [kDa] | [1%] | Id. No. |
| R495H | R495H_{S2}¹ | 59.23 | 15.92 | 1 |
| R495H/K321N | K321N | 59.23 | 15.92 | 2 |
| R495H/K321N/H145L | K321N/H145L | 59.25 | 15.92 | 3 |
| R495H/I5N | I5N | 59.40 | 15.88 | 4 |
| R495H/F31Y | F31Y | 59.42 | 16.12 | 5 |
| R495H/L34Q | L34Q | 59.42 | 15.87 | 6 |
| R495H/M53T/P55T | M53T/P55T | 59.38 | 15.88 | 7 |

¹R495H_{S2} is based on the Cerezyme ® sequence (R495H) but produced in S2 cells instead of CHO cells. All other listed constructs are based on the Cerezyme ® sequence, exclusively produced in S2 cells
²molecular weight and extinction coefficient calculated including BIP (S2 cell protein secretion) signal sequence

Microbiological Methods

The following chapter covers the microbiological methods to express the in this thesis produced and characterized hGCase variants. The cultivation as well as the expression of protein in the S2 cell system was mainly conducted by Daniela Hügin (SMR, Lead Discovery; see section 2.7).

Transient Transfection

All suggested mutants were prepared for the transient transfection in Schneider S2 cells (Schetz & Shankar, 2004). The plasmids with the genotype pExpreS2_1-A-BIP-hGCase(1-497, X, R495H)-GS-Sor-8×His (X=modification of different suggested hGCase variants; table 2.4) were transiently transferred into S2 cells and first expressed without integration into the S2 genome. Abbreviation “1-A” reveals a concatemer, indicating multiple copies of one DNA sequence. “BIP” is the protein secretion signal peptide for insect S2 cells. Via a glycine-serine linker (GS), a sortase recognition sequence is linked to the polyhistidine tag, including eight histidine residues (His8-tag), fused to the C-terminus of the protein. The cells were prepared in ExCell420 media (Sigma-Aldrich) and grown overnight with a cell number of 66.9×106 cell/ml and a measured viability of 99%. All cultures were cultivated in darkness using Climo-Shaker ISF 1-X incubators (Kuhner). The initial culture volume was 20 ml. After incubation overnight at 27° C., 120 rpm, 50 mm shaking radius under the exclusion of CO2 and humidity, the cells were diluted in media (ExCell420) to 6.0×106 cells/ml (99% viability). The transfection reagent ExpreS2 TR5x (Invitrogen) was pre-diluted in media (15 μl in 1 ml media) and vortexed for 1-2 s. The plasmid DNA (5 μg) was added to the mixture. The solution was vortexed again and stored at RT. In parallel, the prepared S2 cell culture was further diluted to a cell density of 4.0×106 cells/ml. 1 ml of prepared S2 culture was pipetted to each prepared DNA-TR5x mixture. The solution was gently mixed and incubated for 72 h at 27° C., 120 rpm, 50 mm shaking radius under the exclusion of CO2 and humidity.

Polyclonal Cell Lines

After incubating the DNA-cell solutions for 72 h at 27° C. after transient transfection, 1.2 ml of the original culture was replaced by media including 1.5 mg/ml zeocin (final concentration). Zeocin is a broad-spectrum glycopeptide antibiotic, effective against most bacteria, fungi, plants, yeasts and animal cells. By selecting cells after zeocin resistance, one can select after cells with the target gene being fully integrated into the host cell genome. When integrated, the gene becomes replicated, thus will be expressed and passed on due to cell division. In total, 22 days of selection were completed with a media exchange conducted every 3-4 days (replacing 1.2 ml by ExCell420 media containing 1.5 mg/ml zeocin). After selection, the stable cell lines were cultivated with a working volume of 30 ml.

Small-Scale Cultivation for hGCase Mutant Screening (30 ml Working Volume)

Small-scale cultures (30 ml) were prepared for every construct. As reference, R495H_{S2} and the Amicus mutant K321N were also freshly cultivated to maintain the same conditions for better comparison in further experiments. For the 30 ml cultures, already in-culture cells with related constructs were taken as inoculation culture. The cell number was determined and diluted to 5×106 cells/ml inoculation concentration. The cells were cultivated at 27° C., 120 rpm with a shaking radius of 50 mm, without CO2 addition as well as under the exclusion of humidity. The cells were roughly splitted twice a week, leading to a total cultivation time of 72-96 h. For a first mutant screening (see section 3.1.1), 1-2 ml of each variant culture was taken and stored at −20° C. for further use (final cell densities: 50-60×106 cells/ml, viability: ˜98%), respectively.

Pilot-Scale Cultivation of Selected hGCase Mutants (5 L Working Volume)

For the pilot-scale cultivation of the selected mutants, 5×1 L cultures were prepared. 30 ml pre-cultures of each hGCase variant were diluted to a cell number of 5×106 cells/ml and passaged to 100-300 ml culture volume. After reaching high enough cell densities, 5×106 cells/ml were further transferred to a final working volume of 1 L. After inoculating 5×1 L for each construct, the cultures were cultivated at 27° C., 90 rpm and 50 mm radius for 96 h before harvesting. Cultivation was performed in darkness under the exclusion of CO₂ and humidity. After 96 h, the cells were harvested by centrifugation (Avanti JXN-26 centrifuge with rotor JS-5.3, Beckman Coulter) with 4000×g for 20 min at 4° C. The cell pellets were discarded; the supernatants were transferred in 1 L cell culture bottles and frozen at −80° C. until used for purification.

Examination of Protein Expression after Cultivation

Western Blots were done to test for protein expression after the transient transfection as well as after establishing stable cell lines (done by Daniela Hügin, SMR, data not shown). Anti-His 6/9 HRPO antibody (in-house produced) was used as detection antibody recognizing the His8-tag fused to the C-terminus of the protein sequences (data not shown). To check for hGCase expression within the final pilot-scale cultures, SDS-PAGE and Western Blot were done as described in section 2.3.4. Here, 0.6 μg total protein (determined after Bradford, section 2.3.2) were loaded in each well for the SDS-PAGE and Western Blot. Additionally, the activity of the culture samples was measured to confirm expression (section 2.3.5).

Protein Biochemical Methods

The following chapter covers all protein biochemical methods that have been applied to identify, purify and finally characterize hGCase variants.

Determination of General Biochemical Properties

Certain general biochemical properties as the molecular weight (MW), isoelectric point (IP) and the extinction coefficient (E 1%) were determined using the web interface SAWI (Sequence Analysis Web Interface, Bioinformatics, Hoffmann-La Roche) by inserting the related gene sequence of a protein.

Determination of Soluble Protein Content after Bradford

To be able to measure the overall protein content not only in cell culture samples but also within purified samples, the standard Bradford assay was used (Bradford, 1976). With the Bradford assay, soluble protein can be detected due to a shift of the absorption maximum to λ=660 nm. This is due to the complexation of the added Coomassie-based dye (Coomassie Brilliant Blue G-250; Pierce™ 660 nm Protein Assay Reagent) in acidic environment with the cationic, covalent side chains of proteins. The increasing signal at λ=660 nm directly correlates with the protein content, photospectrometically detectable. The assay was done following the instructions provided by the vendor (Pierce™ 660 nm Protein Assay, Thermo Scientific, USA). To 10 μl of sample, 150 μl of Pierce™ 660 nm Protein Assay Reagent were added and incubated for 5 min in darkness at RT under gentle movement. Detection was conducted at a wavelength of λ=660 nm with the SpectraMax i3 plate reader (Molecular Devices). The software SoftMax Pro 6.4 was used for visualization and evaluation. Albumin Standard (Thermo Scientific, USA) was used as reference protein (standard curve see appendix I, figure I.1). All measurements were done in triplicate at least.

Determination of Protein Concentration Via NanoDrop

With the NanoDrop, the protein concentration of purified samples was determined. Limitation here is the applicability only for pure protein. The extinction coefficient (E 1%) was determined as described in section 2.3.1. In this work, the NanoDrop2000 Spectrophotometer (Thermo Scientific) was used, including the software NanoDrop2000/2000c. For the measurement, 2 μl of the protein sample were applied. Related solution buffers served as blank. The calculated 260/280 nm ratio should be in a range of ˜0.5 to ensure high protein purity.

SDS-PAGE and Western Blot

SDS-Polyacrylamide Gel Electrophoresis

Sodium dodecyl sulfate polyacrylamide gel electrophoresis is in common use to separate proteins and further determine their molecular weight by separation. For the sample preparation, LDS sample buffer (4×) (Invitrogen), reducing agent (10×) (Invitrogen) and protein sample (5 μg total protein determined after Bradford) are mixed and denatured for 5 min at 95° C. 10 μl of sample as well as marker were loaded in each well (˜0.6 μg total protein). In this work, NuPAGE™ 4-12% Bis-Tris gels (Invitrogen) are used with a 5% (v/v) MOPS/DPBS running buffer (table 2.3). Electrophoresis was carried out applying a constant voltage of 200 V to the gel chamber (Novex Mini-cell; BioRad Criterion™ cell). SeeBlue®Plus2 Prestained Standard (Invitrogen) served as marker. InstantBlue™ (Coomassie-based staining solution, Expedeon, Ltd.) was used as developing solution, H2Obidest. was used as destaining solution. For imaging the gels, the Amersham™ Imager 600 (Amersham Biotech) was used including the related software.

Western Blot

For the evaluation via Western Blot, SDS-PAGE gels were prepared as mentioned in section 2.3.4. iBlot® Gel Transfer Stacks Nitrocellulose, Regular/Mini (Invitrogen) were used as blotting tools, including the stacking layers for the blot. iBlot™ (Invitrogen) served as blotting device. The SDS-PAGE gel was gently removed from the chamber after the run and transferred to the nitrocellulose membrane. A Whatman paper was wetted in H2Obidest. and put on top of the gel. After removing air bubbles in between the stack, the program (P3) of the blotting device was started and run for 7 minutes. The blotted nitrocellulose membrane was transferred to the blocking solution I (see table 2.7) and incubated for 1 h. After removing the excess blocking solution, the membrane was incubated for another hour in blocking solution II, including anti-His 6/9 HRPO antibody (1:10000; in-house produced; table 2.7). After washing the blot several times with DPBS-Tween (0.05% (v/v)) buffer, the detection solutions A (Luminol enhancer solution, Amersham) and B (Peroxide solution, Amersham) were combined at a ratio of 1:1 and immediately dispensed on the membrane. The blot was developed for 5 min and detected by Amersham™ Imager 600 (Amersham Biotech) applying different exposure times.

Determination of GCase Activity Using an Artificial Substrate

The enzymatic activity (EA) of GCase was measured using an artificial substrate resorufin-β-glucopyranoside (res-β-glc; Sigma-Aldrich). Glucocerebrosidase is a hydrolase (EC 3.2.1.45 (BRENDA enzyme database)) catalyzing the conversion of naturally occurring glucosphingolipids, including glucosylceramide (GlcCer) or glucosylsphingosine (GlcSph) into glucose and ceramide/sphingosine, respectively.

In case of the artificial substrate res-β-glc (Sigma-Aldrich), GCase recognizes the glucose moiety and metabolizes the molecule to glucose and resorufin. Resorufin can be spectraphotometrically excited at a wavelength of λ=535 nm and emits light at λ=595 nm.

The enzymatic activity was evaluated by a res-β-glc standard curve. The substrate was diluted in DMSO with a stock concentration of 10 mM. For the substrate standard curve, a res-β-glc concentration range of 0.1-500 μM was prepared and incubated with high protein concentrations of reference protein (R495H_{CHO}, 100 μg/ml) for 60 min to ensure a full turnover of substrate. For each substrate concentration, the emitted signal (in RFU=relative fluorescent unit) at λ=595 nm after 8 min of incubation was taken for the final plot of the standard curve due to undefined decreasing RFU signal effects occurring after long incubation times. The standard curve was fitted with a second order polynomial (quadratic) equation (GraphPad Prism7), following:

y=b0+b1x+b2x² (1)

Using the standard curve, the given signal in RFU can be translated into enzymatic activity (EA). The EA in this work is defined as the conversion of 1 μmol res-β-glc in one minute by a defined enzyme concentration and can be calculated as follows:

$${EA}_{t o t a l} = \frac{{c}_{res .}}{t} \cdot F \cdot V \qquad ( 2 )$$

EAtotal: total enzymatic activity [U]

cres.: concentration of converted res-β-glc [μM]

t: incubation time [min]

F: dilution factor [-]

V: total volume of measured sample [L]

For all further GCase characterization experiments including the activity assay, 20 μM substrate concentration, 30-40 min incubation time and protein concentrations ranging from 25-100 nM were set as final assay settings to ensure measurements in the linear range. The overall DMSO concentration within the assay was kept at 0.5% (v/v) or even below, since DMSO influences the fluorescent signal with higher concentrations (experimentally tested, data not shown; confirmed by (Urban et al., 2008). Therefore, GCase activity buffer+1% (v/v) DMSO was also used for dilutions (table 2.9). The plate reader SpectraMax i3 (Molecular Devices) including the analysis software SoftMax Pro 6.4 was used for all activity measurements within this work. The program settings were kept the same for all activity runs: kinetic run, measurement temperature: 37° C., PMT Gain: high; flashes per read: 6; read height: 0.73305 mm from plate.

Quantification of hGCase with AlphaLISA

The AlphaLISA approach is a simple and effective way to determine protein quantity in related samples. Advantage of this approach is the additional applicability for crude extract measurements. The principle is based on an amplified luminescent proximity assay, where the donor bead (streptavidin-coated) and acceptor bead are brought into close proximity via two conjugated antibodies capturing the same analyte (sandwich assay). By exciting the donor bead at a wavelength of λ=680 nm, released singlet oxygens trigger a cascade of reactions near the acceptor bead, resulting in chemiluminescence, detectable at a wavelength of λ=615 nm. The analytes (f.c.: 10 nM, normalized to R495H_{CHO} concentration), the biotinylated antibody (Ms mAb anti-hGCase 1/23, in-house production, f.c.: 1 nM) and the anti-hGCase acceptor beads (labeled with Ms mAb anti-hGCase 1/17, in-house production, f.c.: 20 μg/ml) are combined, mixed and incubated for 4 h at RT with 150 rpm in darkness. After incubation, donor beads (streptavidin beads, f.c.: 40 μg/ml) were added and incubated for another hour at RT with 150 rpm in darkness before detection. All measurements were done in triplicate. R495H_{CHO} (Genzyme Corp.™) served as reference protein in all measurements, enabling the protein concentration calculation with the AlphaLISA. Detection was done using Paradigm SpectraMax Microplate Reader (Molecular Devices).

Purification and Quality Screening of the hGCase Variants

This chapter covers the purification strategy of in S2 cells produced hGCase variants and the subsequent methods and approaches that have been applied to evaluate and verify protein quality and quantity after purification. For all purification steps, FPLC ÄKTA™ Pure (GE Healthcare Bio-Science), including the flexible fraction collector F9-C was used (if not other mentioned). The UV-signal was detected at a wavelength of λ=280/260 nm for all purification steps. Selected mutants experienced the same purification strategy and were treated equally during the entire purification procedure to ensure comparability. Purification of Amicus mutants K321N and K321N/H145L and reference R495H_{S2} were done in preliminary works (data not shown). The software Unicorn™ 7.3 was used for purification analysis and evaluation.

Purification Strategy of hGCase Variants

Capture step: Affinity chromatography with Concanavalin A (Con A) column Three liter culture supernatant of the variants I5N, F31Y, L34Q and M53T/P55T were first filtered through a 0.22 μm PES membrane sterile filter (Steritop® vacuum filtration system, Millipore, Merck), respectively to remove residual cells and large particles, before loading onto a HiTrap Con A 4B column (1.6 cm×2.5 cm, GE Healthcare Bio-Sciences; column volume (CV): 3×5 ml=15 ml). The resin of this column consists of the protein concanavalin A (Con A) coupled to Sepharose® 4B. Concanavalin A represents a tetrameric metalloprotein, which is widely used for glucoprotein, glucolipid and polysaccharide purifications since the metalloprotein is lectin (carbohydrate-binding protein), allowing to purify after different glycosylation patterns (GE Healthcare Life Sciences, 2019). The column was equilibrated with Con A buffer A (20 mM TRIS/HCl, 0.5 M NaCl, 1 mM MnCl2, 1 mM CaCl₂, 0.02% (v/v) NaN3, pH 7.4; table 2.6) for 5 CV with a flow rate of 5 ml/min. The filtered supernatant was loaded with a flow of 1.8 ml/min. After loading, the column was washed with buffer A for 10 CV (flow: 5 ml/min). By first applying a linear gradient (0-100% Buffer B (20 mM TRIS/HCl, 0.5 M NaCl, 1 mM MnCl2, 1 mM CaCl₂), 0.02% (v/v) NaN3+0.5 M Methyl α-D-mannopyranoside, pH 7.4; table 2.6)) for 15 CV followed by a continued elution (100% Buffer B) for 10 CV, the protein was eluted and collected (total volume ˜360-370 ml). Elution fractions with highest activity were pooled (section 2.3.5). 1 ml of every step during the Con A purification was shock frozen on dry ice and retained at −80° C. for further analysis.

2nd Step: Immobilized Metal-Chelate Affinity Chromatography (IMAC)

For the second purification step, a HisTrap™ HP column (GE Healthcare, 1.6×2.5 cm) with CV=5 ml was used. The column contains a Ni Sepharose High Performance (HP) affinity resin that interacts with the His8-tag of the proteins, enabling a specific and high-resolution purification. The interaction takes place by chelating groups that have been coupled to the sepharose resin. The groups are further precharged with nickel ions, interacting with the histidine tag of proteins. The used HisTrap HP column was first equilibrated for 2 CV with IMAC buffer A (50 mM HEPES/NaOH, 0.5 M NaCl, 10 mM imidazole and 0.02% (v/v) NaN3, pH 7.6; table 2.6)). After equilibration, the protein sample (Con A pool) was loaded onto the column with a flow of 0.2 ml/min (loaded overnight). Following, the column was washed for 10 CV with buffer A (flow: 5 ml/min). Step elutions for 5 CV with 4%, 8% and 10% IMAC buffer B (50 mM HEPES/HCl 0.5 M NaCl, 0.5 M imidazole and 0.02% (v/v) NaN3, pH 7.6; table 2.6) were performed to elute weakly bound protein, according to the increasing imidazole concentration. A final gradient elution for 15 CV with a followed continued elution step of 100% buffer B (0.5 M imidazole for 5 CV) was included to elute the target protein and stronger bound proteins. Peak fractions were tested on activity, elution fractions with highest activity were pooled (section 2.3.5). 1 ml of every step during the IMAC purification was shock frozen on dry ice and retained at −80° C. for further analysis.

3rd step: Hydrophobic Interaction Chromatography (HIC)

As sample preparation for the hydrophobic interaction chromatography (HIC), 0.5 M KCl was added to the IMAC pool of the second step. The salt was slowly added under constant stirring to ensure equal distribution, avoiding concentration peaks within the pool. In this work, a Toyopearl Butyl-M 650 1.1 ml HIC column (Tosoh Bioscience, 1.0×1.4 cm) was self-packed with a CV of 1.1 ml. The resin contained hydroxylated methacrylic polymer beads that are functionalized with butyl ligand groups (Tosoh Bioscience LLC, 2019). Initially, the column was equilibrated for 1 CV with HIC buffer A (20 mM MES/HCl, 0.5 M KCl and 0.02% (v/v) NaN3; table 2.6). The sample was loaded with a flow of 1 ml/min. After loading, the column was washed to remove unbound protein (flow: 1 ml/min for 7 CV). The elution was done using HIC buffer B (table 2.6), containing 80% (v/v) ethylene glycol due to the strong interaction of target protein with the HIC column resin. For the elution, a linear gradient was performed (15 CV), followed by a continued elution with 100% buffer B for 10 CV. Peak fractions were tested on activity with elution fractions showing highest activity and protein content being pooled (section 2.3.5). The pool was dialyzed against the end buffer (20 mM histidine, 140 mM NaCl, pH 6; table 2.6). 1 ml of every step during the HIC purification was shock frozen on dry ice and retained at −80° C. for further analysis.

Dialysis and Storage of Purified Protein

After the last purification step, the sample was dialyzed against the end buffer (table 2.6). For the dialysis, Slide-A-Lyzer® Dialysis Cassettes, 10000 MWCO (Thermo Scientific) were used. The dialysis was done overnight at 4° C. under continuous stirring. For a complete buffer change, a dialysis buffer volume of at least 200× the initial sample volume is recommended. Following, the sample was filtrated through a Millex® Syringe-driven filter unit (Millipore, Merck), ensuring sterile conditions. For concentration purposes, Amicon® Ultra-15 centrifugal filters, 10000 NMWL (Millipore, Merck) concentrators were used. The sample was centrifuged at 3600×g (MegaStar 3.OR, VWR) until reaching the desired volume/concentration. The final samples were aliquoted, shock frozen on dry ice and stored at −80° C. until use.

Purchased Cerezyme® (R495H_{CHO}) as Benchmark Reference

Commercially available Cerezyme® (R495H_{CHO}; imiglucerase; Genzyme Corp.™) was purchased and initially dialyzed against the end buffer (20 mM histidine, 140 mM NaCl, pH 6.0; table 2.6). The buffer change should ensure closer comparability between the in CHO produced R495H_{CHO} and the in S2 produced hGCase mutants in this work. Yet, the production systems of the molecules differ, thus still not directly comparable. R495H_{CHO} served as reference molecule already in clinical use.

Characterization of hGCase Variants

In the context of this work, several approaches were done to closely characterize the hGCase variants in their biochemical properties. For this, the variants were tested on purity, yield and specific enzyme activity (herein spec. EA). Further, performance indicators as the kinetic parameters KM, Vmax, kcat and kcat/KM were calculated and IC50 values were determined using two different inhibitors (reversible and irreversible). The activatability of the molecules by adding phospholipids (here: phosphatidylserine) was investigated as well as the thermal stability. A possible shift in the pH optimum was evaluated by performing a pH screen. An analytical SEC and sedimentation velocity-analytical ultracentrifugation (SV-AUC) were used to gather information about the oligomeric state of the molecules. Finally, the uptake, activity and substrate reduction in human H4 glioblastoma cells was tested to evaluate performances in a cellular-based assay setup.

Thermal Stability of hGCase Mutants

The thermal stability was determined performing two different thermal stress approaches as described in the following.

Thermal Shift Assay Via nanoDSF Measurement

The thermal shift assay was performed via nanoDSF (differential scanning fluorimetry) measurements using Prometheus NT.Plex nanoDSF (NanoTemper Technologies). The measurement was initialized using the software PR.ThermoControl. Prometheus can precisely characterize samples by measuring the thermal unfolding, chemical denaturation and aggregation within one single run (NanoTemper Technologies, 2019). By gradually heating the sample until reaching 95° C., the protein unfolds. Thereby, the chemical environment of tryptophan residues changes (change in intrinsic tryptophan fluorescence of protein due to more aqueous environment), causing a red-shift in fluorescence (tryptophan fluorescence emission shift). By plotting the fluorescence ratio 350/330 nm against the temperature, the melting temperature Tm can be determined. Calculating the first derivative of raw data gives a peak curve with its maximum indicating the unfolding temperature Tm.

For the sample preparation, a concentration of 0.5 mg/ml was adjusted for the hGCase mutants. For variant I5N and R495H_{S2}, protein concentrations of 0.075 mg/ml (I5N) and 0.06 mg/ml (R495H_{S2}) were applied due to low protein yields. 15 μl total sample volume was uptaken in each capillary of the capillary rack (Prometheus NT.Plex Capillary Chips, NanoTemper) and placed in the device. The excitation power was adjusted to 20-80%, depending on the applied protein concentration. The gradually heating was set to 3° C./min until reaching 95° C. (20° C. was set as starting temperature). The evaluation and calculation of the first derivative was done using the NanoTemper PR.ThermoControl software, the final evaluation and graphing was done using the PR.StabilityAnalysis software.

Heat Inactivation at 55° C.

Another approach to test the mutants for thermal stability is the irreversible heat inactivation at 55° C. (Futami et al., 2017; Zale & Klibanov, 1986). As initial screening to select for most promising candidates, this thermal stability approach was chosen (section 3.1.1). For this, supernatant samples of each construct were taken from the 30 ml S2 cell cultures, respectively (section 2.2). The protein concentration was not adjusted. The samples were incubated for 40 min in total at 55° C. (PCR Cycler Mastercycler gradient, Eppendorf) using PCR plates (FrameStar®96 well skirted PCR plate, 4titude Ltd.). Samples were taken after 5, 7, 10, 15, 20, 30 and 40 min for residual activity measurements. The activity assay was performed as described in section 2.3.5. The initial activity of each mutant was set to 100% before heating. Residual activities were calculated in regard to the initial activity. Data points were fitted using the one phase decay fit by GraphPad Prism7 following equation 3:

y=(y₀−Plateau)·e^{(-k·x)}+Plateau (3)

where y0 represents the starting point of decay (100% initial activity before heating), the plateau is set to 0% (end point; 0% residual activity) and k represents the rate constant equal to the reciprocal of the x-axis units.

For the heat inactivation experiment of purified protein (see section 3.3.3), hGCase protein samples were prepared with a final concentration of 50 nM, diluted in hGCase activity buffer, pH 6.0 (table 2.9) and incubated for 5 and 10 minutes in the waterbath (Julabo 5A, Julabo) at 55° C.

pH Screening of hGCase Variants

A pH optimum screening was done to observe a possible shift in the pH spectrum of the new variants due to structural modification of the enzymes. Therefore, three different buffers were used, covering a pH range of pH 3.5 to 8. For the screening, buffer substances were chosen after the use for purification buffers. Therefore, following buffers were prepared: Citric acid buffer (20 mM, pH 3.5-5), MES buffer (20 mM, pH 6-6.5) and HEPES buffer (20 mM, pH 7-8). The final protein concentration used for this assay was 75 nM. The activity assay was done as described in section 2.3.5 (table 2.11), merely the buffer was varied.

Determination of the Kinetic Parameters

The kinetic parameters were examined using the artificial substrate resorufin-β-glucopyranoside (Sigma-Aldrich). For the determination, final concentrations of substrate ranging from 4-500 μM were tested. The assay followed the activity assay instructions in section 2.3.5. 25 nM final protein concentration was used for each construct. The incubation time was set to 30 min to ensure measurements in the linear signal range. The kinetics were evaluated and plotted according to Michaelis-Menten (Cornish-Bowden, 2013; Johnson, 2015). KM, Vmax was calculated by GraphPad Prism7 applying the Michaelis-Menten fit equation provided by the program. The equation model follows:

$$y = \frac{{V}_{max} \cdot x}{( {K}_{M} + x )} \qquad ( 4 )$$

with:
KM: Michaelis-Menten constant [μM]
Vmax: maximum velocity of reaction [μM/min]

KM is defined as the substrate concentration at which the reaction reaches half the maximum velocity. Therefore, the equation for calculating KM follows:

K_{M}=½Vₘₐₓ (5)

with: KM: Michaelis-Menten constant [μM]
Vmax: maximum velocity of reaction [μM/min]

The maximum velocity of a reaction is reached when all available enzyme molecules are complexed in an enzyme-substrate complex (ES), simplified as follows:

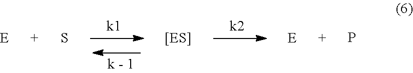

The enzyme (E) and the substrate (S) form an enzyme-substrate complex (ES), where the catalytic reaction of the enzyme takes place, either converting the substrate to a product (P) or the collapse of the ES complex, leading to the release of unmodified substrate and enzyme. The rate constant k either describes the association of E and S (bimolecular) or the disassociation/back reaction to enzyme (E) and substrate (S) (unimolecular). Since the formation of an enzyme-product (EP) complex and the following collapse to (E) and (P) happens with a high reaction rate, the (EP) step can be neglected. Also, since the substrate concentration usually exceeds the enzyme concentration in such an experiment, the formation to (ES) and further to (P) and (E) is more likely than the back reaction to (E) and (S). Therefore, the basic Michaelis-Menten equation can be expressed as:

$$V = {V}_{max} ( \frac{[ S ]}{[ S ] + {K}_{M}} ) \qquad ( 7 )$$

with: V=initial velocity of the reaction [μM/min]
Vmax: maximum velocity of reaction [μM/min]
[S]: substrate concentration [μM]
KM: Michaelis-Menten constant [μM]

The kinetic parameter kcat is defined as the catalytic constant, representing the turnover number of substrate to product per time unit (here per second) (Eisenthal et al., 2007). kcat can be calculated as follows:

$${k}_{c a t} = \frac{{V}_{max}}{{[ E ]}_{tot .}} \qquad ( 8 )$$

with:
kcat: turnover number [min-1]
Vmax: maximum velocity of reaction [μM/min]
[E]tot.: total enzyme concentration [nM]

Another kinetic key parameter to describe the catalytic efficiency of enzymes is the parameter kcat/KM, measured in [μM−1*s−1] in this work (Eisenthal et al., 2007).

Determination of IC50 of hGCase Mutants

The inhibition of hGCase was tested using a reversible, competitive inhibitor isofagomine (IFG, in-house produced) and an irreversible inhibitor, conditurol-β-epoxide (CBE, Sigma-Aldrich).

Inhibition of hGCase Using Isofagomine (IFG)

IFG is a competitive, reversible inhibitor of hGCase, interacting with the active pocket of the enzyme (Powe et al., 2006).

To calculate the IC50, a concentration range of 0.002-100 μM IFG was tested. IFG was prepared with a stock concentration of 1 mM in DMSO. The final enzyme concentration was 25 nM. After adding the inhibitor to the enzyme, the solution was pre-incubated for 10 min at RT before adding the substrate (20 μM) for the activity measurement. The activity assay setup is shown in table 2.

TABLE 2
hGCase activity assay scheme for IC50 determination with IFG.

| Substance | Concentration | Blank |
|---|---|---|
| Protein¹ | 25 nM | — |
| Inhibitor² | 0.002-100 μM | 0.002-100 μM |
| Buffer³ | X | X |

Pre-incubation for 10 min at RT

| | | |
|---|---|---|
| Substrate⁴ | 20 μM | 20 μM |

Incubation for 40 min at 37° C.
Kinetic measurement at 37° C. using SpectraMax i3 (Molecular Devices)
Excitation: λ = 535 nm; Emission: λ = 595 nm
¹protein here refers to the sample containing the purified GCase
²here isofagomine (IFG)
³GCase activity buffer, X: volume adjustable
⁴artificial substrate resorufin-β-glucopyranoside

The evaluation and IC50 determination were done using GraphPad Prism7. hGCase activity with no inhibitor added was set to 100% relative enzyme activity. In case of evaluating the raw data via GraphPadPrism7, the applied inhibitor concentrations were first transformed to log 10×-values plottet on the x-axis. Further, the raw data measured with the activity assay in RFU was normalized to the highest activity value within one data set (equals 100%). 100% signal was defined as 10-12 μM IFG (=no inhibitor added). Data points were fitted and IC50 values were determined using log(inhibitor) vs. response-variable slope (four parameters) (GraphPadPrism7) following equation 9:

$$y = Bottom + \frac{Top - Bottom}{( 1 + 1 {0}^{( {logIC}_{50} - x ) * HillSlope}} \qquad ( 9 )$$

where top and bottom represent the curve plateaus in the unit of the y-axis. The HillSlope describes how steep the curves are. A HillSlope of −1.0 indicates a normal range, −2.0 would indicate a steeper curve course.

Inhibition of hGCase Using Conditurol-β-Epoxide (CBE)

CBE is an irreversible inhibitor of glucocerebrosidases which covalently binds to the active site of the enzyme (Liou et al., 2006).

A stock solution of 100 mM CBE in H₂Oultrapure was prepared. For IC50 determination, CBE concentrations ranging from 0.04-2500 μM were tested. Again, the final enzyme concentration was 25 nM. After adding the inhibitor, the enzyme was pre-incubated for 10 min before initializing the activity measurement by adding the substrate (20 μM). The final activity assay is shown in table 3 follows:

TABLE 3
hGCase activity assay scheme for IC50 determination with CBE.

| Substance | Concentration | Blank |
|---|---|---|
| Protein¹ | 25 nM | — |
| Inhibitor² | 0.04-2500 μM | 0.04-2500 μM |
| Buffer³ | X | X |

Pre-incubation for 10 min at RT

| | | |
|---|---|---|
| Substrate⁴ | 20 μM | 20 μM |

Incubation for 40 min at 37° C.
Kinetic measurement at 37° C. using SpectraMax i3 (Molecular Devices)
Excitation: X = 535 nm; Emission: X = 595 nm
¹protein here refers to the sample containing the purified hGCase
²here conditurol-β-epoxide (CBE)
³GCase activity buffer (table 2.9), X: volume adjustable
⁴artificial substrate resorufin-β-glucopyranoside

IC50 evaluations were done in the same manner as described for IFG (see section above). The IC50 calculations were done using GraphPad Prism7 following equation 9.

Activatability of hGCase Mutants by Phosphatidylserine

Negatively charged lipids as phosphatidylserine are known to stabilize and activate the enzyme usually in combination with Saposin C (SAP-2), since GCase is naturally interacting with the membrane of the lysosomes in vivo. For the assay, 25 nM protein and 4 μM phosphatidylserine (Avanti; 10 mM stock solution dissolved in chloroform) were tested. The activity assay is shown in table 4:

TABLE 4
Activity assay scheme for phosphatidylserine activation measurement.

| Substance | Concentration | Blank |
|---|---|---|
| Protein¹ | 25 nM | — |
| Activator² | 4 μM | 4 μM |
| Buffer³ | X | X |

Pre-incubation for 10 min at RT

| | | |
|---|---|---|
| Substrate⁴ | 20 μM | 20 μM |

Incubation for 40 min at 37° C.
Kinetic measurement at 37° C. using SpectraMax i3 (Molecular Devices)
Excitation: X = 535 nm; Emission: X = 595 nm
¹protein here refers to the sample containing the purified hGCase
²here phosphatidylserine
³GCase activity buffer (table 2.9), X: volume adjustable
⁴artificial substrate resorufin-β-glucopyranoside

After adding the lipid to the enzyme, the solution was pre-incubated for 10 min at RT before adding the substrate and initializing the enzymatic reaction. The hGCase activity without added phosphatidylserine was set to 100% relative enzyme activity. The increase in activity was expressed as X-fold the initial activity without activator.

Performance of hGCase Mutants on Human H4 Glioblastoma Cells in Uptake, Activity and Glucosylsphingosine (GlcSph) Reduction

The experiments and evaluations of the hGCase variants on human H4 glioblastoma cells (H4 cells) were exclusively done by Nadia Anastasi (NRD) and Iris Ruf (SMR, Hit Qualification Assays), thus only briefly mentioned in this chapter.

Human H4 glioblastoma cells (H4 cells) were taken as human neuronal model to test purified hGCase variants in a cell-based setup. H4 wild-type cells and untreated H4 GBA-KO cells (knock-out of related GCase GBA gene) were taken as references. After seeding (40000 cells/well) and incubating them for ˜24 h, the GBA-KO cells were treated with 1, 10 and 100 nM of target protein sample and incubated for either 2 h (for uptake and GCase activity measurements) or 48 h (for substrate reduction measurements) at 37° C. with 5% CO2 and constant humidity. After cell-lysis (GCase activity buffer (table 2.6)+0.1% (w/v) Triton X-100+phosphatase (Phospho stop Roche in-house)+protease inhibitor (cOmplete EDTA-free)), the uptake of the enzymes into the cells was evaluated by measuring the hGCase concentration via AlphaLISA (section 2.3.6) as well as the activity after the uptake (section 2.3.5). Samples for substrate reduction determination were hand over to Iris Ruf (SMR, see section 2.7). Due to the loss of the GCase related GBA gene in H4 GBA-KO cells, the natural substrate glucosylsphingosine (GlcSph) accumulates. The reduction of GlcSph levels due to hGCase treatment was evaluated via LC/MS by Iris Ruf (SMR).

Data Evaluation

Calculations and evaluations of data were mainly done using Microsoft Excel 2010 and GraphPad Prism7 using the according fit equations as mentioned within the related sections in more detail All measurements were done in triplicate at least with final results given in mean values +/−standard deviation.

Results

Quality Analysis of Final hGCase Batches Via HP-RPC

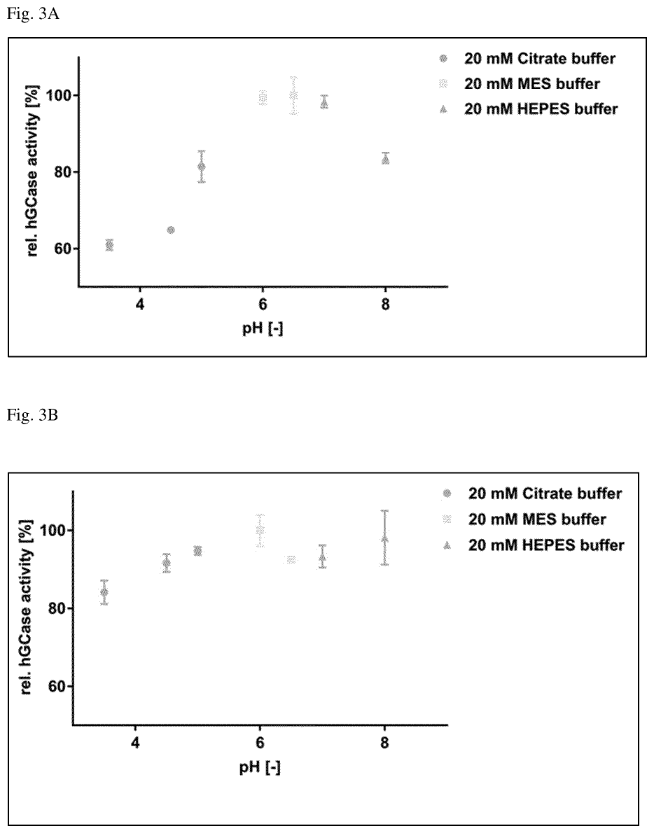

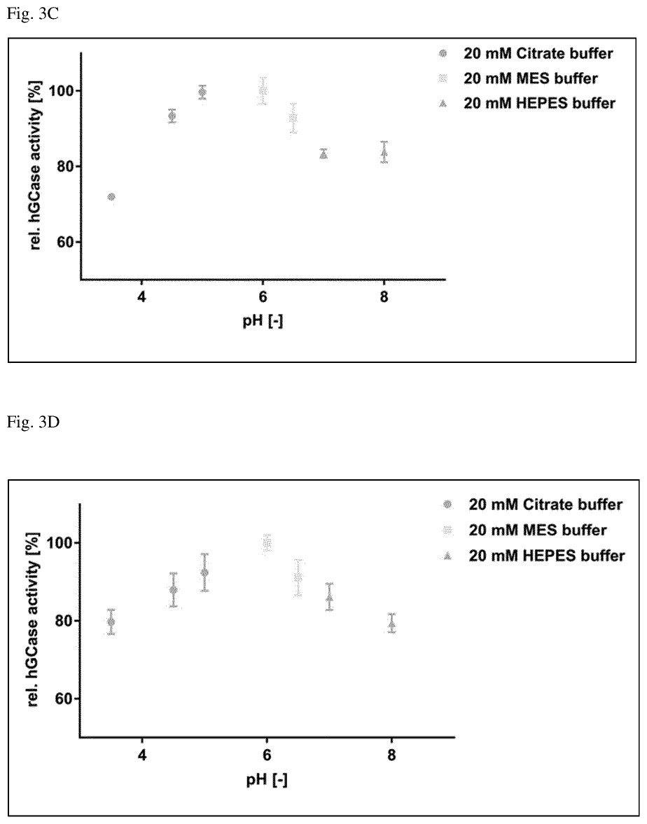

To more precisely investigate the quality of final protein batches, high performance-reversed phase chromatography (HP-RPC) was used to separate, identify and quantify components in the final hGCase pools. The column Poroshell 300SB-C8 (1.0×75 mm, 1 CV=0.06 ml, Agilent Technologies) was used. Due to the difference in applicable pressures (50-350 bar) and the smaller dimensions in columns and particle size (2-50 μm), the HP-RPC possesses a higher resolving power, beneficial for separating and identifying substances within a sample mixture even at low concentrations. FIG. 3.8 shows the representative HP-RPC chromatogram of mutant M53T/P55T.

The HP-RPC chromatogram showed a UV signal peak (λ=280 nm) at a retention time of 1.966 minutes with a signal intensity of ˜50 mAU for M53T/P55T. The protein was eluted applying ˜70% mobile phase B (table 2.6). Due to the injection of pure reference protein R495H_{CHO}, the retention time of GCase molecules could be determined, confirming ˜1.9 min. Slight changes in retention times compared to reference R495H_{CHO} (2.011 min) could be explained by the fused His8-tag for the hGCase variants, missing with R495H_{CHO} as well as the differing production system (S2 cells vs. CHO cells). By integrating the signal peak, the area under the curve could be calculated and compared to injected reference protein in terms of protein quantity. However, with the use of HP-RPC herein, purity determinations of final protein batches were in focus rather than quantity. With no other peak observable (FIG. 3.8), mutant M53T/P55T elucidated 100% purity with no detectable impurities or degraded protein. HP-RPC was run for each purified protein batch and evaluated in table 5.

TABLE 5
Summary of determined HP-RPC parameters of all purified mutants
as well as chosen references.

| | | | | K321N/ | | | | M53T/ |
|---|---|---|---|---|---|---|---|---|
| | R495H_{CHO} | R495H_{S2} | K321N | H145L | I5N | F31Y | L34Q | P55T |
| retention time | 2.011 | 1.972 | 1.956 | 1.964 | 1.966 | 1.982 | 1.980 | 1.966 |
| [min] | | | | | | | | |
| purity | 100 | 100 | 100 | 86 | 90 | 90 | 95 | 100 |
| [%] | | | | | | | | |

All molecules roughly showed the same retention time, indicating the hGCase molecule to be similarly purified. R495H_{S2}, K321N and the mutant M53T/P55T indicated no impurities, whereas K321N/H145L revealed two additional peaks in the HP-RPC chromatogram with retention times of 1.6 and 1.8 min, indicating a more impure protein batch. The same pattern could be seen for variant I5N, F31Y and L34Q, resulting in reduced purity.

Thermal Stability of hGCase Mutants

As mentioned above, two hGCase variants used as references were reported to have increased thermal stability compared to the Cerezyme® molecule imiglucerase (Hung, 2015). To identify potentially stabilized variants, two different approaches were applied to test for thermal stability from different points of view.

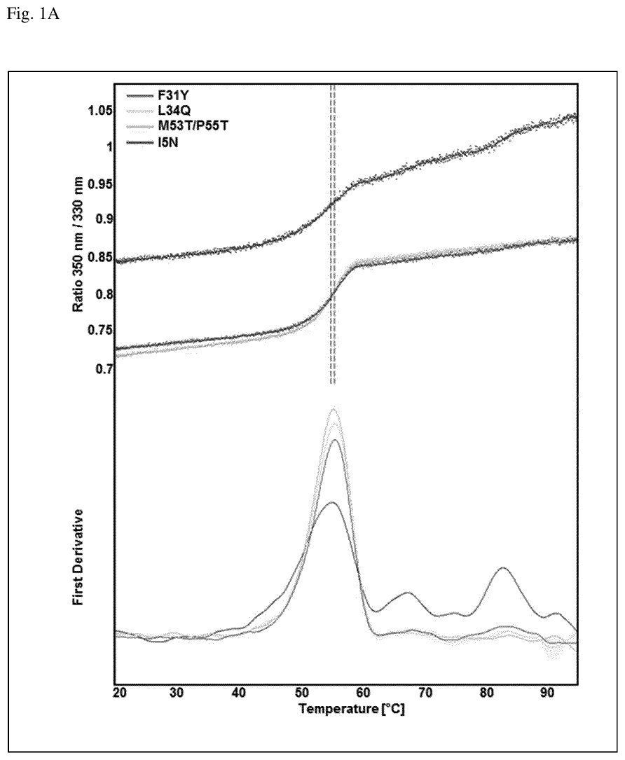

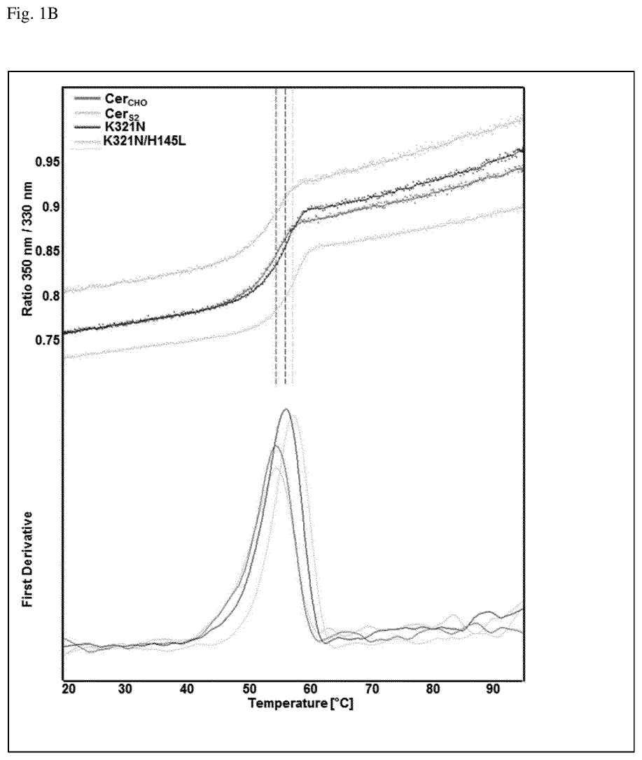

On the one hand, a successively increasing temperature gradient was applied to measure melting temperatures of protein molecules via nanoDSF (differential scanning fluorimetry) measurement using Prometheus NT.Plex (NanoTemper Technologies, 2019). The sample is gradually heated by increasing the temperature until reaching 95° C. Thereby, the protein unfolds and tryptophan residues experience a change in chemical environment (exposure to a more aqueous environment), which changes the intrinsic tryptophan fluorescence of the protein, causing a red shift in the fluorescent spectrum (350/330 nm=tryptophan fluorescence emission shift) (Schubert, 2015). In FIG. 1, typical melting curves of such an unfolding experiment are shown.

In the upper section of the graphs (FIGS. 1 (A) and (B)), the raw data (progression curves) measured with nanoDSF is plotted, indicating the shift in the fluorescence ratio 350/330 nm during the course of the measurement with increasing temperature. The calculation of the first derivative, giving peak curves (lower section of graphs in (A) and (B)), was done according to default settings of the Software PR.StabilityAnalysis (table 2). Peak maxima (thermal unfolding transition midpoint) indicate the unfolding temperature Tm, where half of the proteins are unfold (Schubert, 2015). Table 6 summarizes the unfolding temperatures of each construct.

Table 6: Summarized unfolding temperatures Tm of tested hGCase mutants via nanoDSF measurement using Prometheus NT.Plex.

TABLE 6
Summarized unfolding temperatures Tm of tested hGCase mutants via nanoDSF
measurement using Prometheus NT.Plex.

| | | | | K321N/ | | | | M53T/ |
|---|---|---|---|---|---|---|---|---|
| | R495H_{CHO} | R495H_{S2} | K321N | H145L | I5N | F31Y | L34Q | P55T |
| Tₘ | 55.2 ± 0.0 | 54.6 ± 0.0 | 56.2 ± 0.1 | 57.3 ± 0.0 | 54.8 ± 0.4 | 55.1 ± 0.1 | 55.2 ± 0.0 | 55.3 ± 0.1 |
| [° C.] | | | | | | | | |

All mutants exhibited higher or equal thermal stability compared to R495H_{S2} (54.6° C.), with the majority indicating ˜55° C. Mutant I5N had the highest deviation and might be explained by the low concentrations applied due to initial low yields, resulting in measurement variability. Only the reference molecules K321N and K321N/H145L (Amicus mutants) showed a significant increase in thermal stability with 1.5-3° C. increase in Tm.

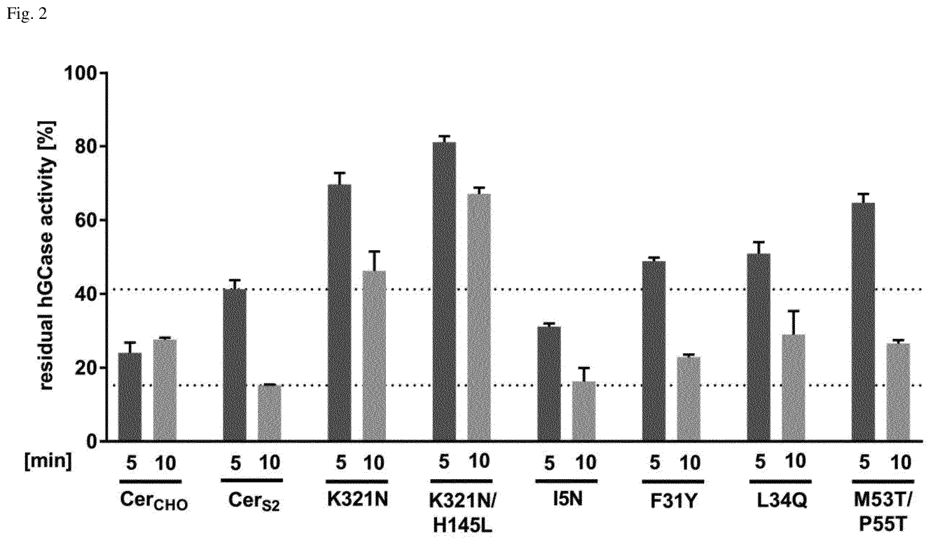

The second approach was aimed to test residual GCase activity after thermal inactivation at a fixed temperature. With this additional approach, the focus was set on activity-based changes with increased incubation time (Futami et al., 2017; Zale & Klibanov, 1986). Here, samples were incubated for 5 and 10 minutes in the waterbath at 55° C. Residual GCase activities were measured with the initial activity set to 100% (before heating). FIG. 2 depicts calculated residual activities for tested mutants after incubation.

The activity-based approach gave further information about the enzyme's ability to maintain its activity even under thermal stress. Based on two different incubation time points, one could extrapolate how fast and to which extent different hGCase variants lose their activity. All mutants showed higher residual activities measured after 5 and 10 min of incubation compared to R495H_{S2}, except for variant I5N and R495H_{CHO}. Amicus mutant K321N and K321N/L145H showed significantly higher residual activities with both incubation times, corresponding to the nanoDSF measurements (table 3.6). Regarding the novel tested candidates, variant M53T/P55T showed highest residual activity after 5 min of incubation (65%), which is then reduced to only 27% residual activity after 10 min of incubation, similar to F31Y and L34Q. I5N indicated poor performance with a residual activity of 31% after 5 min and only 16% after 10 min. R495H_{CHO} seemed to rapidly lose its initial activity but retained residual activity with further incubation. However, the results for R495H_{CHO} should be confirmed by repeating the measurements with the same experimental setup.

In general, both approaches revealed similar tendencies, with Amicus variants indicating molecules optimized for thermal stability as already reported, whereas the new mutants performed either moderately better or similar to R495H_{S2}.

Determination of pH Optimum Shift Due to Mutations

A possible pH optimum shift due to structural modifications should be examined. Since the GCase enzyme originally occurs within the lysosome, its natural pH optimum is reported to be between pH 4.7-6.0 (Lieberman et al., 2007; Liou et al., 2006; Tan et al., 2014). This could change when including slight modifications in the structure of the molecule. Thus, the mutants were tested within a pH range of 3.5 to 8 for enzymatic activity. FIG. 3 shows the pH spectra of four selected hGCase mutants.

The pH profiles of the mentioned mutant consistently showed a pH optimum of pH 6 (100% relative activity). Generally, the tested hGCase variants tended to retain their activity rather with higher pH values (7-8 with ˜80-90% relative activity) than with lower pH between 3.5 and 4 (60-80% activity), observable for most of the variants. Exception here was mutant M53T/P55T and K321N/H145L with a more bell-shaped pH spectrum (FIG. 3). R495H_{CHO} lost 40% of its activity when incubated at pH 3.5 (20 mM citric acid buffer) compared to e.g. R495H_{S2} and M53T/P55T with remaining 80% relative activity within the same pH. Vice versa, variant I5N indicated 75% activity at pH 3.5 but turned completely inactive at pH 8. Variant F31Y and L34Q implied similar pH spectra, revealing ˜80% relative activity at low pH (3.5) with increasing activity until reaching pH 6 (100%) and further retaining almost 85-95% of activity with higher pH. Overall, no variant (except for I5N at pH 8) dropped below 50% residual activity over the whole tested pH range. For the consolidation of data, the buffer screen needs to be repeated with buffers possessing nearly the same charges on the conjugated base and ionic strength held constant during pH studies as well as overlapping pH ranges.

IC50 Determination with Natural Inhibitor Isofagomine and Irreversible Inhibitor Conditurol-β-Epoxide

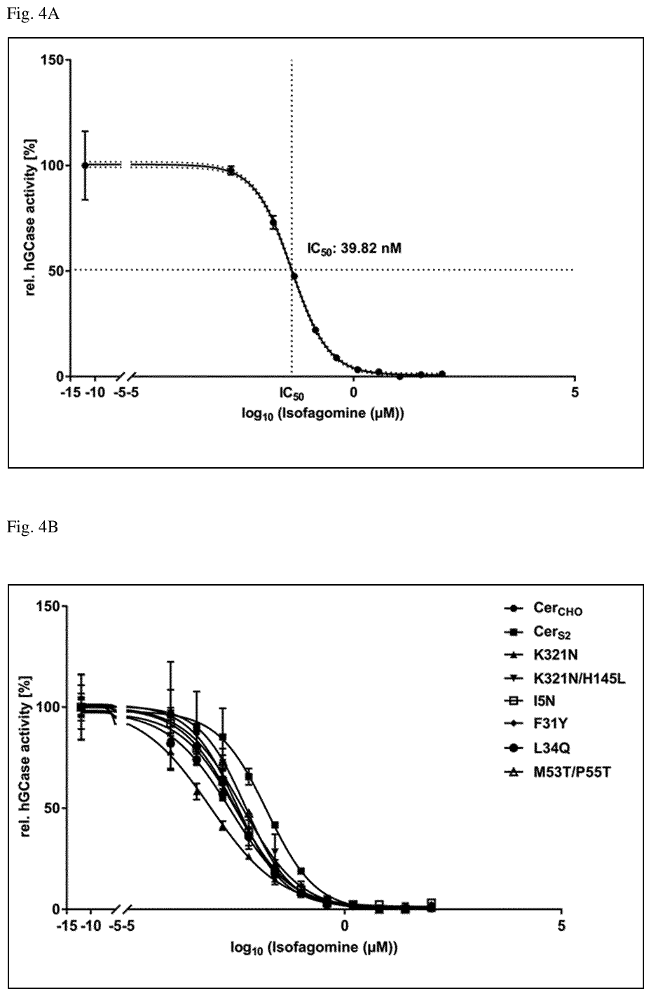

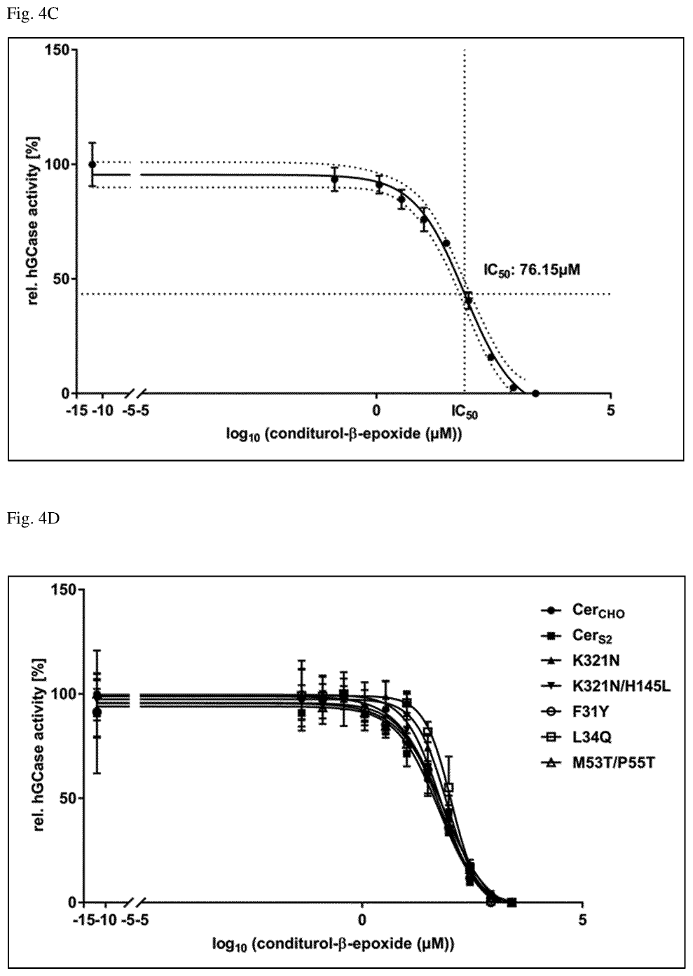

To determine whether the active binding site of an enzyme is compromised or altered, IC50 values were calculated for active site inhibitor and compared. In this work, the natural occurring GCase inhibitor isofagomine (IFG) and conditurol-β-epoxide (CBE) were used. IFG is known as a reversible inhibitor, interacting with the active site of the GCase enzyme, whereas CBE is an irreversible inhibitor of GCase (Powe et al., 2006). The IC50 of an enzyme is defined as the concentration of an inhibitor, where the response, in this case the enzyme activity, is reduced by half (Kalliokoski et al., 2013). Both assays were conducted as described in section 2.5.6. The inhibitor profiles of M53T/P55T using IFG and CBE as well as the IC50 profiles of all residual mutants combined are shown in FIG. 4.

The raw data was plotted by transforming the applied IFG/CBE concentration to a logarithmic scale (x-axis, FIG. 4) and by normalizing the activity signal with highest signal of each data set to 100% (defined with apparent inhibitor concentration of 10-12 μM, equal to no inhibitor). IC50 values were calculated using GraphPad Prism7 following equation 9. An IC50 of ˜40 nM was calculated for mutant M53T/P55T with IFG and an IC50 of ˜76 μM with CBE (FIGS. 4 (A) and (C)). All determined IC50 values are shown in table 7.

TABLE 7
Summary of IC50 values for hGCase variants determined with IFG and CBE.

| | | | | K321N/ | | | | M53T/ |
|---|---|---|---|---|---|---|---|---|
| | R495H_{CHO} | R495H_{S2} | K321N | H145L | I5N | F31Y | L34Q | P55T |
| IC₅₀ | 27 ± 6 | 17 ± 3 | 28 ± 3 | 35 ± 11 | 26 ± 3 | 28 ± 3 | 22 ± 4 | 40 ± 2 |
| IFG | | | | | | | | |
| [nM] | | | | | | | | |
| IC₅₀ | 60 ± 3 | 55 ± 3 | 28 ± 2 | 35 ± 3 | n.d.¹ | 60 ± 3 | 106 ± 1 | 76 ± 3 |
| CBE | | | | | | | | |
| [μM] | | | | | | | | |

¹n.d. = not determined

For the calculated IC50 with IFG, all values, except for R495H_{S2}, were roughly in a similar range between ˜20 and 40 nM. R495H_{S2} showed a slightly lower IC50 with 17 nM. A second inhibitor should further verify proper functionality of the active binding site of the new hGCase mutants. R495H_{CHO}, R495H_{S2} as well as F31Y and M53T/P55T indicated similar IC50 values of ˜60-70 μM measured with CBE, considering the standard deviations. K321N and double mutant K321N/H145L showed more potent CBE IC50 values of 28 μM and 35 μM but differ with two times lowered IC50 compared to R495H_{CHO} and R495H_{S2}. Contrarily behaved variant L34Q with a measured IC50 of 106 μM with CBE, which was almost doubled compared to the reported IC50 with R495H_{CHO}. IC50 determination for I5N with CBE could not be performed due to shortage of purified sample. Generally, a higher deviation in IC50 values was observed across the molecules when tested with CBE, which should be followed up with additional enzyme kinetic measurements and structural testings.

Comparison of Kinetic Parameters of the hGCase Mutants

Determining enzyme kinetic key parameters gives solid information about the efficiency and efficacy of an enzyme and how certain mutations can impact its functionality. For the determination and comparison of the enzymatic performances, KM, Vmax and kcat as well as kcat/KM were calculated for the different hGCase variants, respectively (Eisenthal et al., 2007). The according assay was performed as described in section 2.5.5, all measured with the artificial substrate res-3-glc. KM and Vmax determination for representative mutant M53T/P55T was done applying the Michaelis-Menten curve fit (GraphPad Prism7; see equation 4) as shown in the following.

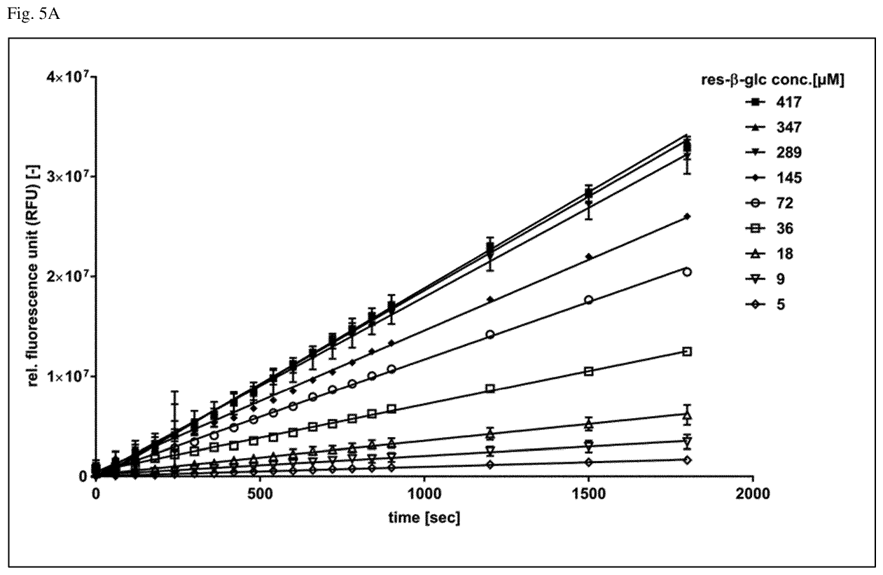

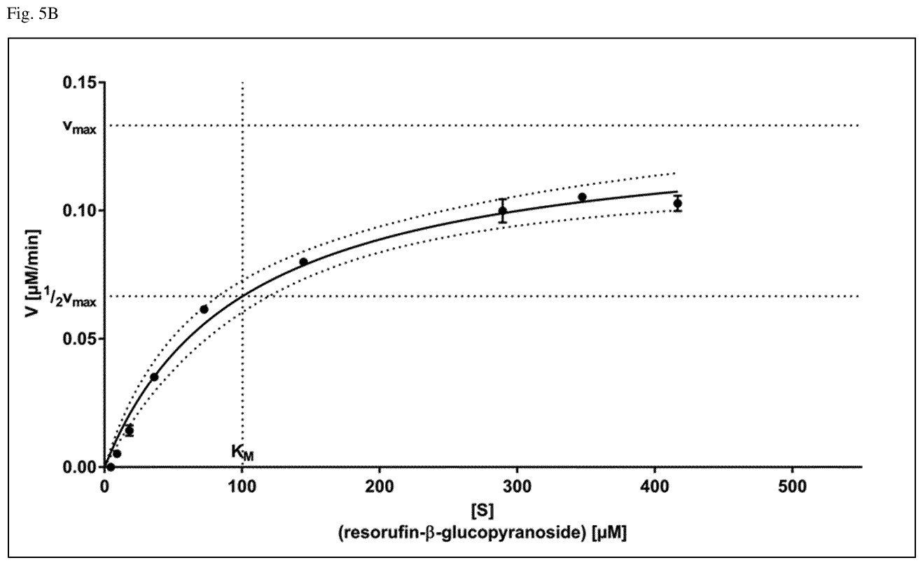

The kinetic raw data is depicted in FIG. 5 (A) with progression curves following pseudo first order in a linear measurement range with a good reproducibility and low variance. The slopes of the progression curves can be replotted, with velocities V [μM/min] plotted as a function of substrate concentration [S] applied, resulting in a typical course of a Michaelis-Menten curve (Cornish-Bowden, 2013; Johnson, 2015). The calculated kinetic key parameters are summarized in table 8.

TABLE 8
Summary of key kinetic parameters of tested hGCase variants.

| | | | | K321N/ | | | | M53T/ |
|---|---|---|---|---|---|---|---|---|
| | R495H_{CHO} | R495H_{S2} | K321N | H145L | I5N | F31Y | L34Q | P55T |
| K_{M} [μM] | 130.7 ± | 188.6 ± | 130.7 ± | 156.3 ± | 118.4 ± | 96.6 ± | 143.4 ± | 100.3 ± |
| | 21.1 | 45.5 | 22.8 | 32.3 | 24.4 | 15.2 | 17.2 | 15.8 |
| Vₘₐₓ | 0.14 ± | 0.08 ± | 0.14 ± | 0.13 ± | 0.14 ± | 0.11 ± | 0.17 ± | 0.13 ± |
| [μM/min] | 0.01 | 0.01 | 0.01 | 0.01 | 0.01 | 0.01 | 0.01 | 0.01 |
| k_{cat}¹ | 0.09 | 0.05 | 0.09 | 0.09 | 0.09 | 0.07 | 0.11 | 0.09 |
| [s⁻¹] | | | | | | | | |
| k_{cat}/K_{M} | 0.69 | 0.27 | 0.69 | 0.58 | 0.76 | 0.72 | 0.77 | 0.90 |
| [μM⁻¹ * s⁻¹ * | | | | | | | | |
| 10⁻³] | | | | | | | | |

¹protein concentration used: 25 nM for all constructs

With regard to the deviations calculated for KM, all variants were approximately in a similar range with KM ˜100-180 μM. Poor performance showed R495H_{S2} with an apparent KM of 188.6±45.5 μM and Vmax=0.08±0.01 μM/min. When calculating Vmax, all mutants indicated higher Vmax than R495H_{S2} with L34Q showing highest maximum velocity (0.17±0.01 μM/min). The turnover efficiency of an enzyme can be expressed by kcat. It represents the catalytic constant for the conversion of substrate to product, considering a single, representative active site for a given enzyme concentration (see equation 8, (Eisenthal et al., 2007)). Thus, kcat values should correlate with Vmax by trend to a certain degree (equation 8). A more defined parameter reveals kcat/KM, often referred to as specificity constant and widely used to compare efficiencies of enzymes. Here, K321N, I5N, F31Y and L34Q as well as R495H_{CHO} showed similar performances with kcat/KM ˜0.7 μM−1*s−1*10-3, since deviations in KM or Vmax values certainly influence this ratio. I5N showed a surprisingly good kinetic performance, whereas only poor results in spec. EA could be evaluated after purification. R495H_{S2} depicted poorest performance in enzyme kinetics with almost two times lower values calculated for kcat and kcat/KM compared to the other hGCase variants (table 8). Interestingly, double mutant K321N/H145L performed rather poor with an apparent high KM (158.3 μM) and decreased kcat/KM (0.58 μM−1*s−1*10-3) but indicated decent spec. EA after purification. Overall, all variants showed increased kcat/KM values compared to R495H_{S2}, even when considering KM deviations, due to better performances in Vmax achieved with each mutant. Notably, variant M53T/P55T showed a good performance with all calculated parameters, resulting in highest apparent kcat/KM value (0.9 μM−1*s−1*10-3) with comparably low KM (100.3±15.8 μM) and moderate Vmax (0.13±0.01 μM/min).

Effect of Lysosomal Membrane Lipid Phosphatidylserine on hGCase

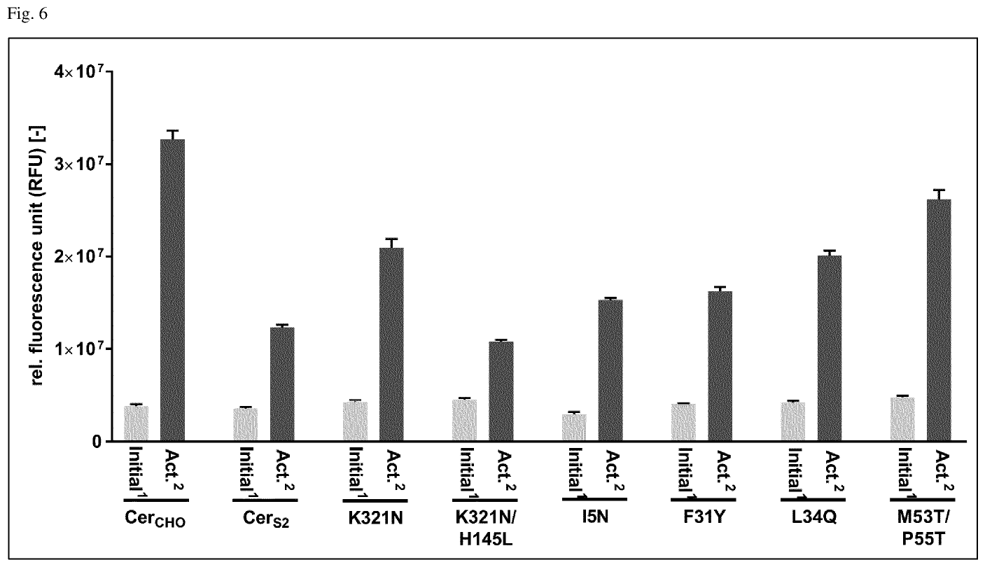

Several physiological substances have been reported to stimulate the activity of glucocerebrosidase. GCase is naturally interacting with the membrane of the lysosomes in vivo, thus negatively charged lipids such as phosphatidylserine are known to stabilize and activate the enzyme alone or in combination with Saposin C (SAP-2) (Qi & Grabowski, 1998; Sun et al., 2002; Vaccaro et al., 1995). Thus, it is essential to preserve these properties when modifying the GCase enzyme structure. To this end, the effect of phosphatidylserine alone was evaluated as first approach. The activity levels with and without added phosphatidylserine are plotted in FIG. 6.

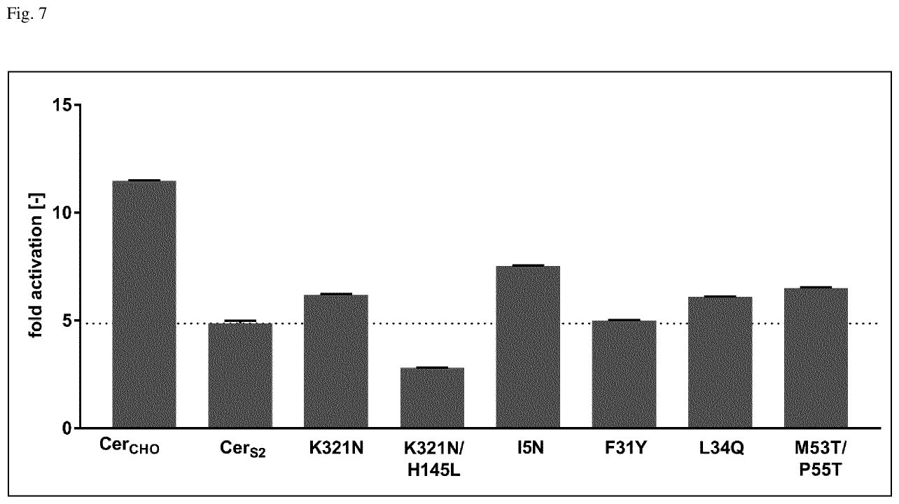

By translating the data into fold increase of the initial activity, a more conclusive statement can be drawn (FIG. 7).

All tested hGCase variants could be activated by the phospholipid. Amicus double mutant K321N/H145L showed the least activation (˜2.8-fold). Except for K321N/H145L, all variants exhibited equal or higher activation levels than R495H_{S2} (4.9-fold). With a 6.5-fold increase for M53T/P55T and 7.5-fold increase for I5N, these two variants showed the best phospholipid stimulatory effects among the novel mutants, however I5N had the least basal activity level (FIG. 6). Interestingly, R495H_{CHO} surpassed all tested variants with an 11.5-fold increased activation after the addition of the phospholipid.

Performance of hGCase Mutants on Human Glioblastoma H4 Cells as Neuronal Cell Model

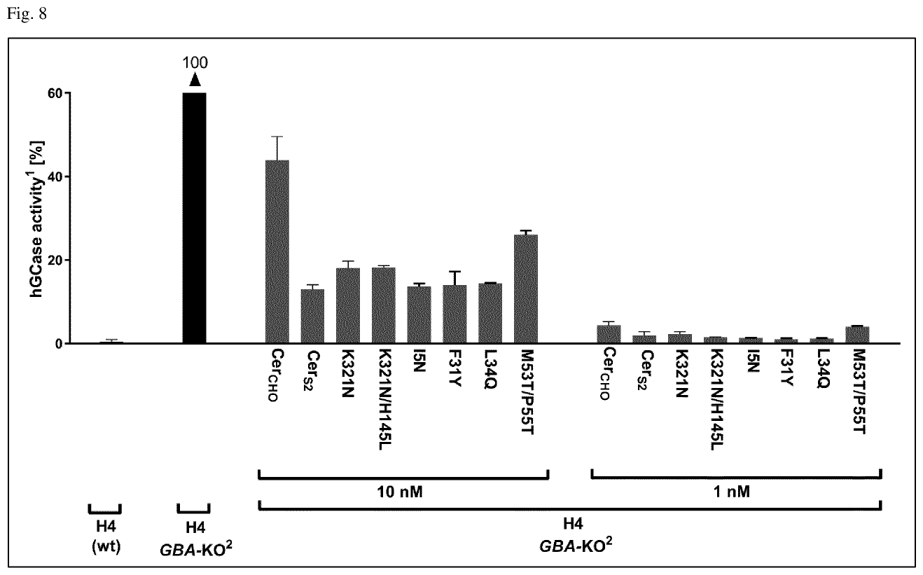

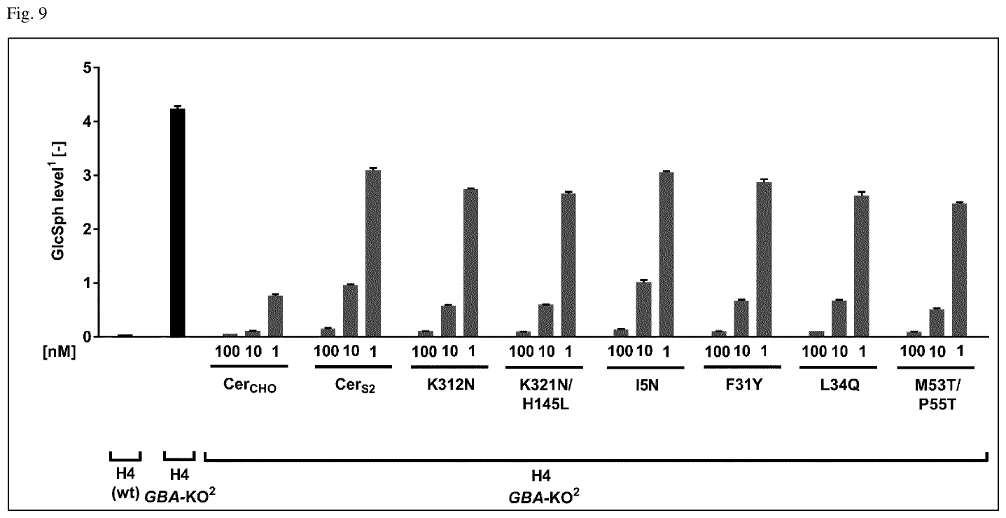

Finally, the cellular uptake, activity and substrate reduction in human neuronal cells were determined to test the mutants' efficacy in a more cellular context. In this work, human H4 glioblastoma wild-type cells (wt) and H4 GBA-KO cells (knock-out of GCase related GBA gene) were used as a Gaucher cell model. H4 GBA-KO cells, which accumulate the natural substrate glucosylsphingosine (GlcSph) due to the loss of the GBA gene, were treated with 1, 10 and 100 nM of hGCase molecules, respectively. Activity and protein concentration (via AlphaLISA) were measured after two hours of incubation after several media wash steps. The reduction of natural substrate GlcSph was analyzed 48 h after cell treatment via LC/MS. The activity measured after uptake into the cells normalized to measured hGCase protein levels is graphed in FIG. 8. The evaluated activities are given as percentage of hGCase activity measured in wt H4 cells, set to 100%.

In general, lower activity levels were measured for all hGCase construct compared to the GCase activity levels measured in H4 wt cells (black bar). R495H_{CHO} showed the highest activity level with 44% normalized to H4 wt GCase activity level with 10 nM protein treatment concentration. All molecules indicated equal or higher activity levels compared to R495H_{S2} (with 10 nM treatment). Double mutant M53T/P55T showed best results with 26% activity compared to H4 wt (10 nM treatment), still not reaching GCase enzyme levels naturally occurring in H4 wt cells (black bar). Roughly the same trend was observed for 1 nM protein treatment concentration although mutant M53T/P55T now indicated similar activity levels to R495H_{CHO} (4%). Importantly, the tested R495H_{CHO} sample was not dialyzed in the usually used end buffer of the S2 produced molecules for this experiment. Thus, it was applied in its original formulation buffer. This could have an impact on activity since additives in the formulation buffer could stabilize the enzyme and improve the overall performance of the molecule.

To determine the enzymes' efficacies, the reduction of accumulated glucosylsphingosine (GlcSph) in H4 GBA-KO cells was investigated. FIG. 9 shows the measured GlcSph levels (measured via LC/MS, normalized to protein levels measured after uptake) according to the treated enzyme concentrations as well as initial substrate levels measured in GBA-KO cells (black bar).

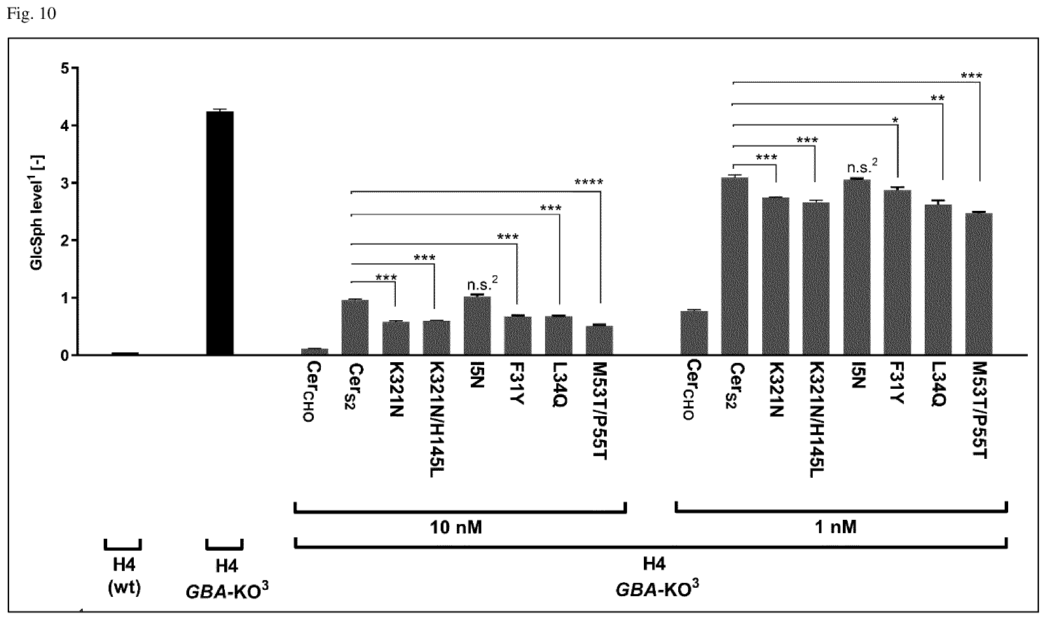

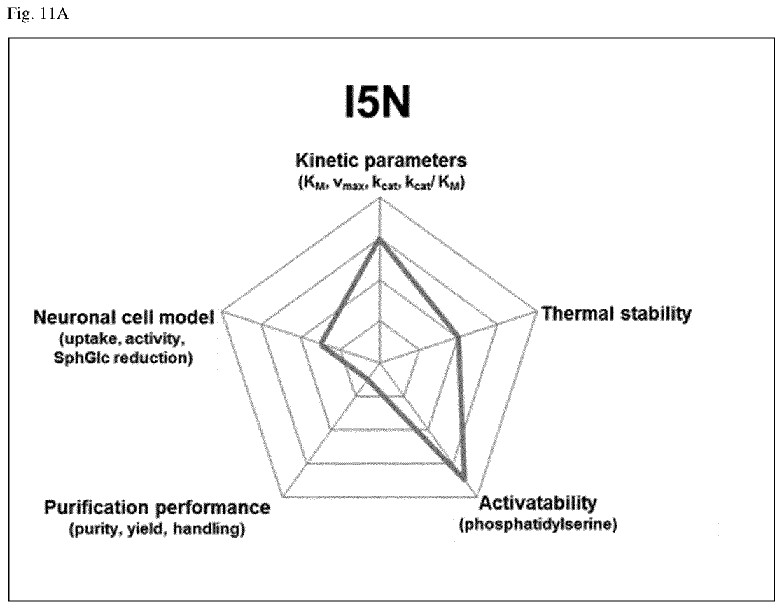

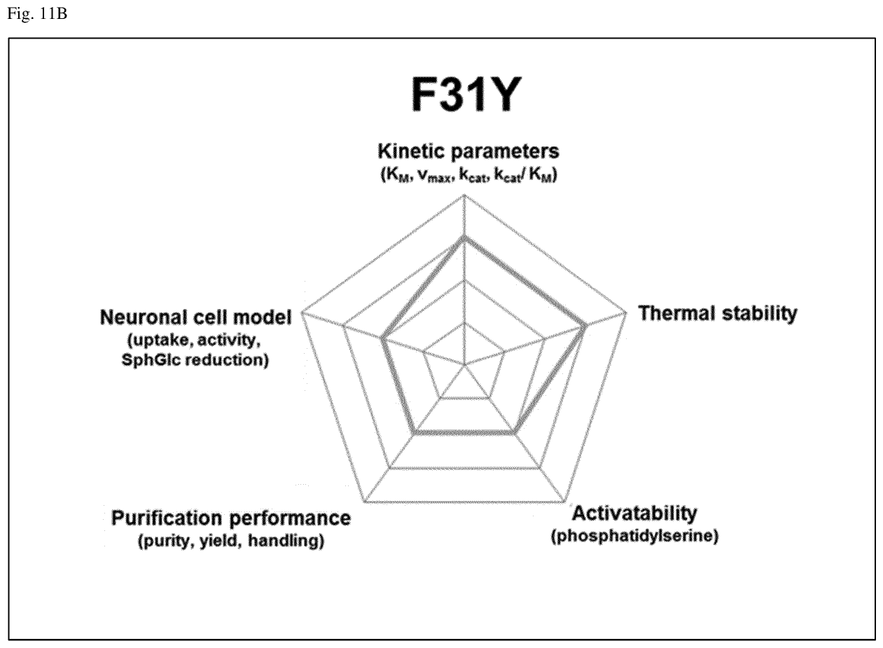

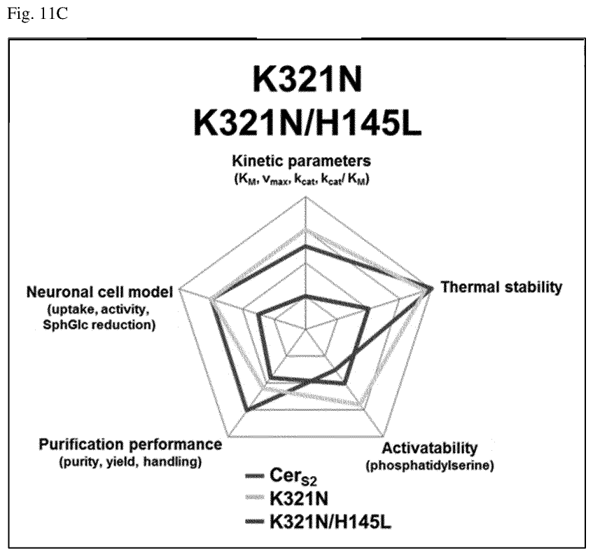

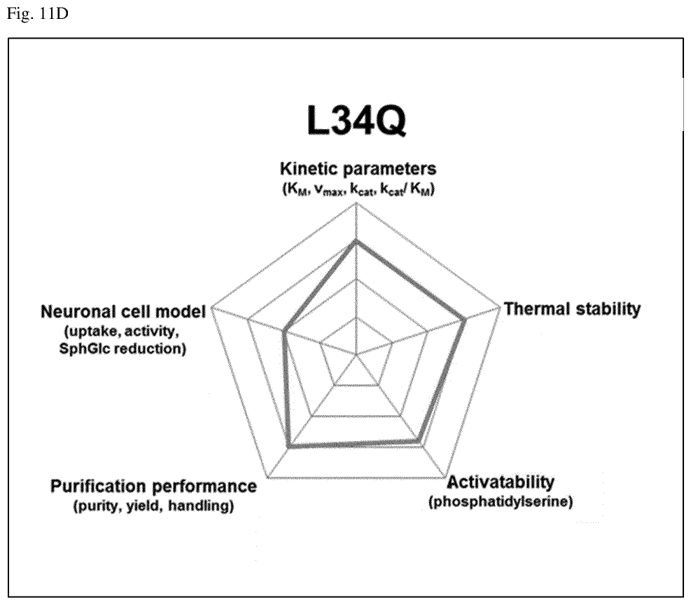

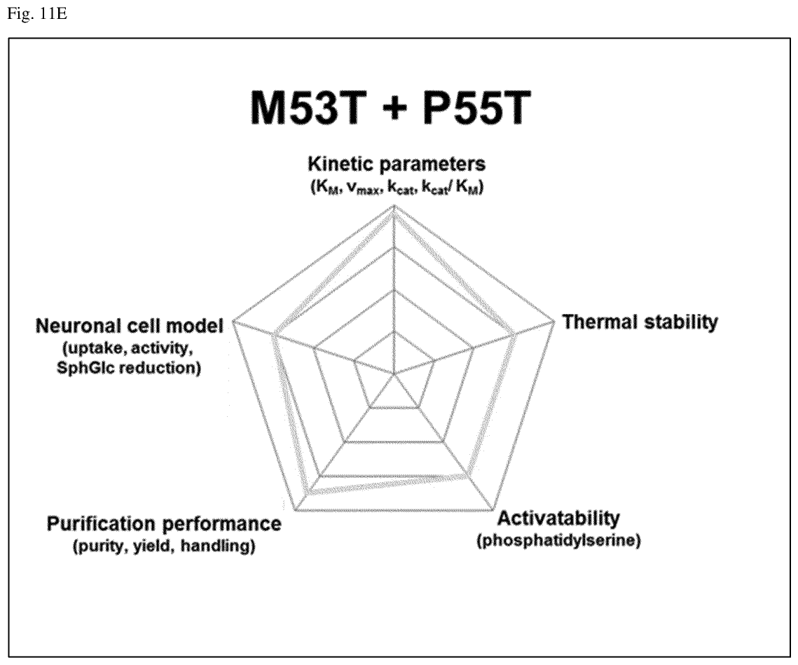

All tested molecules revealed a reduction in GlcSph substrate levels compared to the untreated H4 GBA-KO cells (black bar) with all three tested concentrations, respectively. For better assessment, the data was regraphed in FIG. 10.

With 1 nM enzyme concentration, only a reduction by 1.4 to 1.7 (˜30-40%) in GlcSph levels was achieved (FIG. 10) for all tested constructs. With 10 nM protein treatment, a more conclusive picture could be drawn, still indicating similar tendencies as with 1 nM treatment. Mutants I5N and R495H_{S2} were least efficacious at 10 nM with a reduction by 4 (˜75% reduction) of the GlcSph levels compared to the levels initially measured in H4 GBA-KO cells. Especially for mutant I5N, a discrepancy between the enzyme kinetic performance (table 8) and the herein evaluated performance on H4 cells was observable and needs to be discussed. All mutants (except for I5N) showed a better reduction in natural substrate than R495H_{S2}. Variant M53T/P55T was the best performing molecule tested (except for R495H_{CHO}) with highest GlcSph substrate reduction (8.3-fold reduction=˜88% reduction with 10 nM treatment). The fact that R495H_{CHO} was most efficacious (82% reduction with 1 nM; 97% reduction with 10 nM treatment) might be again due to stabilizing additives or buffer conditions from its original formulation buffer. In future experiments, dialyzed R495H_{CHO} with the same buffer conditions used for the S2 material should be planned, leading to a better understanding of the functional differences.

### REFERENCES

Abrahamov, A., Zimran, A., Varughese, S., Chertkoff, R., Tan, E. S., Giraldo, P., Petakov, M., Gonzalez-Rodriguez, D. E. & Cooper, P. A. (2016). Long-term safety and efficacy of taliglucerase alfa in pediatric Gaucher disease patients who were treatment-naïve or previously treated with imiglucerase. Blood Cells, Mol Dis 68, 163-172. Shire Human Genetic Therapies, Inc.
Alfonso, P., Aznarez, S., Giralt, M., Pocovi, M. & Giraldo, P. (2007). Mutation analysis and genotype/phenotype relationships of Gaucher disease patients in Spain. J Hum Genet 52, 391-396.
Barton, N. W., Furbish, F. S., Murray, G. J., Garfield, M. & Brady, R. O. (1990). Therapeutic response to intravenous infusions of glucocerebrosidase in a patient with Gaucher disease. Proc Natl Acad Sci USA 87, 1913-1916.
Barton, N. W., Brady, R. O., Dambrosia, J. M., Biscegile, A. M., Doppelt, S. H., Hill, S. C., Mankin, H. J., Murray, G. J., Parker, R. I. & other authors. (1991). Replacement therapy for inherited enzyme deficiency-macrophage-targeted glucocerebrosidase for Gaucher's disease. N Engl J Med 324, 1464-1470.
Beutler, E. and Grabowski, G. A. (1994). Gaucher disease. In Metab Mol Bases Inherit Dis, pp. 2641-2670. Edited by C. Scriver, A. Beaudet, W. Sly & D. Valle. McGraw-Hill Medical Publishing company.
Beutler, E. & Grabowski, G. A. (2001). Gaucher disease. In Metab Mol Bases Inherit Dis, pp. 3635-3668. Edited by C. Scriver, A. Beaudet, W. Sly & D. Valle. McGraw-Hill Medical Publishing company.
Beutler, E., Demina, A. & Gelbart, T. (1994). Glucocerebrosidase Mutations in Gaucher Disease. Mol Med 1, 82-92.
Beutler, E., Gelbart, T. & Scott, C. R. (2005). Hematologically important mutations: Gaucher disease. Blood Cells, Mol Dis 35, 355-364.
Bradford, M. M. (1976). A rapid and sensitive method for the quantitation of microgram quantities of protein utilizing the principle of protein-dye binding. Anal Biochem 72, 248-254.
Brady, R. O., Kanfer, J. & Shapiro, D. (1965). The Metabolism of Glucocerebrosides I. Purification and properties of a glucocerebrosidase-cleaving enzyme from spleen tissue. J Biol Chem 240, 39-43.
Butters, T., Dwek, R., van Weely, S., Hrebicek, M., Platt, F., Cox, T., Moyses, C., Lachmann, R., Aerts, J. & other authors. (2000). Novel oral treatment of Gaucher's disease with N-butyldeoxynojirimycin (OGT 918) to decrease substrate biosynthesis. Lancet 355, 1481-1485.
Cornish-Bowden, A. (2013). The origins of enzyme kinetics. FEBS Lett 587, 2725-2730.
Eisenthal, R., Danson, M. J. & Hough, D. W. (2007). Catalytic efficiency and kcat/KM: a useful comparator? Trends Biotechnol 25, 247-249.
Futami, J., Miyamoto, A., Hagimoto, A., Suzuki, S., Futami, M. & Tada, H. (2017). Evaluation of irreversible protein thermal inactivation caused by breakage of disulphide bonds using methanethiosulphonate. Sci Rep 7, 1-10. Springer US.
Gaucher, P. C. E. (1882). De l'épithélioma primitif de la rate. Hypertrophie idiopathique de la rate sans leucémie. Academic thesis. Fac médecine Paris.
Grabowski, G. A. & Horowitz, M. (1997). Gaucher's disease: Molecular, genetic and enzymological aspects. Baillieres Clin Haematol 10, 635-656.
Grabowski, G. A., Barton, N. W., Pastores, G., Dambrosia, J. M., Banerjee, T. K., McKee, M. A., Parker, C., Schiffmann, R., Hill, S. C. & Brady, R. O. (1995). Enzyme Therapy in Type 1 Gaucher Disease: Comparative Efficacy of Mannose-Terminated Glucocerebrosidase from Natural and Recombinant Sources. Ann Intern Med 122, 33-39.
Grabowski, G. A. & Dagan, A. (1984). Human lysosomal β-glucosidase: Purification by affinity chromatography. Anal Biochem 141, 267-279.
Grabowski, G. A., Gatt, S., Kruse, J. & Desnick, R. J. (1984). Human lysosomal β-glucosidase: Kinetic characterization of the catalytic, aglycon, and hydrophobic binding sites. Arch Biochem Biophys 231, 144-157.
Grabowski, G. A. (2008). Phenotype, diagnosis, and treatment of Gaucher's disease. Lancet (London, England) 372, 1263-1271.
Hruska, K. S., LaMarca, M. E., Scott, C. R. & Sidransky, E. (2008). Gaucher disease: Mutation and polymorphism spectrum in the glucocerebrosidase gene (GBA). Hum Mutat 29, 567-583.
Hung, D. (2015). Variant, recombinant, beta-glucocerebrosidase proteins with increased stability and increased retained catalytic activity. U.S. Pat. No. 8,962,564 B2. United States of America.
Johnson, K. A. (2015). Author's personal copy A century of enzyme kinetic analysis, 1913 to 2013. FEBS Lett 587, 2753-2766.
Kalliokoski, T., Kramer, C., Vulpetti, A. & Gedeck, P. (2013). Comparability of Mixed IC50 Data—A Statistical Analysis. PLoS One 8.
Kornhaber, G. J., Tropak, M. B., Maegawa, G. H., Tuske, S. J., Coales, S. J., Mahuran, D. J. & Hamuro, Y. (2008). Isofagomine induced stabilization of glucocerebrosidase. Chembiochem 9, 2643-2649.
Lieberman, R. L., Wustman, B. A., Huertas, P., Powe, A. C., Pine, C. W., Khanna, R., Schlossmacher, M. G., Ringe, D. & Petsko, G. A. (2007). Structure of acid β-glucosidase with pharmacological chaperone provides insight into Gaucher disease. Nat Chem Biol 3, 101-107.
Liou, B., Kazimierczuk, A., Zhang, M., Scott, C. R., Hegde, R. S. & Grabowski, G. A. (2006). Analyses of variant acid β-glucosidases: Effects of Gaucher disease mutations. J Biol Chem 281, 4242-4253.
Powe, A., Steet, R. A., Wustman, B., Kornfeld, S. A., Chung, S. & Do, H. (2006). The iminosugar isofagomine increases the activity of N370S mutant acid beta-glucosidase in Gaucher fibroblasts by several mechanisms. Proc Natl Acad Sci 103, 13813-13818.
Qi, X. & Grabowski, G. A. (1998). Acid β-glucosidase: Intrinsic fluorescence and conformational changes induced by phospholipids and saposin C. Biochemistry 37, 11544-11554.
Van Rossum, A. & Holsopple, M. (2016). Enzyme Replacement or Substrate Reduction? A Review of Gaucher Disease Treatment Options. Hosp Pharm 51, 553-563.
Sato, Y. & Beutler, E. (1993). Binding, internalization, and degradation of mannose-terminated glucocerebrosidase by macrophages. J Clin Invest 91, 1909-1917.
Schetz, J. A. & Shankar, E. P. N. (2004). Protein expression in the Drosophila Schneider 2 cell system. Curr Protoc Neurosci 1-15.
Schneider, I. (1972). Cell lines derived from late embryonic stages of Drosophila melanogaster. J Embryol Exp Morphol 27, 353-65.
Schubert, T. D. (2015). nanoDSF. Precisely revealing protein folding and stability. 2bind molecular interactions.
Sidransky, E., Nalls, M. A., Aasly, J. O., Aharon-Peretz, J., Annesi, G., Barbosa, E. R., Bar-Shira, A., Berg, D., Bras, J. & other authors. (2009). Multicenter Analysis of Glucocerebrosidase Mutations in Parkinson's Disease. N Engl J Med 361, 1651-1661.
Sidransky, E. & Lopez, G. (2012). The link between the GBA gene and parkinsonism. Lancet Neurol 11, 986-998.
Sidransky, E., LaMarca, M. E. & Ginns, E. I. (2007). Therapy for Gaucher disease: Don't stop thinking about tomorrow. Mol Genet Metab 90, 122-125.
Smith, L., Mullin, S. & Schapira, A. H. V. (2017). Insights into the structural biology of Gaucher disease. Exp Neurol 298, 180-190. Elsevier.
Sun, Y., Grabowski, G. A., Qi, X., Kondoh, K. & Qin, W. (2002). Functional Organization of Saposin C. J Biol Chem 271, 6874-6880.
Tan, Y. L., Genereux, J. C., Pankow, S., Aerts, J. M. F. G., Yates, J. R. & Kelly, J. W. (2014). ERdj3 is an endoplasmic reticulum degradation factor for mutant glucocerebrosidase variants linked to Gaucher's disease. Chem Biol 21, 967-976.
Urban, D. J., Zheng, W., Goker-Alpan, O., Jadhav, A., Lamarca, M. E., Inglese, J., Sidransky, E. & Austin, C. P. (2008). Optimization and validation of two miniaturized glucocerebrosidase enzyme assays for high throughput screening. Comb Chem High Throughput Screen 11, 817-24.
Vaccaro, A. M., Ciaffoni, F., Barca, A., Salvioli, R., Tognozzi, D., Scerch, C. & Tatti, M. (1995). pH-dependent Conformational Properties of Saposins and Their Interactions with Phospholipid Membranes. J Biol Chem 270, 30576-30580.
Vunnam, R. R. & Radin, N. S. (1980). Analogs of Ceramide That Inhibit Glucocerebroside. Org Synth 26, 265-278.
Zale, S. E. & Klibanov, A. M. (1986). Why Does Ribonuclease Irreversibly Inactivate at High Temperatures. Biochemistry 25, 5432-5444.
Zimran, A., Altarescu, G., Philips, M., Attias, D., Jmoudiak, M., Deeb, M., Wang, N., Bhirangi, K., Cohn, G. M. & other authors. (2010). Phase ½ and extension study of velaglucerase alfa replacement therapy in adults with type 1 Gaucher disease: 48-month experience 115, 4651-4656.

1. A recombinant human β-Glucocerebrosidase protein, wherein the protein comprises at least one substitution relative to Seq. Id. No. 1, and wherein the substitution is selected from the group consisting of: I5N, F31Y, L34Q, M53T and P55T, and combinations thereof.

2. The recombinant human β-Glucocerebrosidase protein of claim 1, wherein the protein comprises at least two substitutions relative to Seq. Id. No. 1, wherein the two substitutions are M53T and P55T.

3. The recombinant human β-Glucocerebrosidase protein of claim 1 or 2 comprising an amino acid sequence selected from the group consisting of Seq. Id. No. 4-7.

4. The recombinant human β-Glucocerebrosidase protein of claims 1-3, wherein the protein comprises an amino acid sequence set forth in Seq. Id. No. 7.

5. An isolated nucleic acid encoding the human β-Glucocerebrosidase protein of claims 1-4.

6. A vector comprising the nucleic acid sequence of claim 5.

7. A host cell comprising the vector of claim 6.

8. A pharmaceutical formulation comprising the recombinant human β-Glucocerebrosidase protein of claims 1-4.

9. A recombinant human β-Glucocerebrosidase protein of claims 1-4 for use as a medicament.

10. A recombinant human β-Glucocerebrosidase protein of claims 1-4 for use in the treatment of a neurodegenerative disease, in particular Gaucher's disease and Parkinson's disease.

11. A conjugate comprising a human β-Glucocerebrosidase protein of claims 1-4 and a blood brain barrier shuttle.

12. The conjugate of claim 11 for use as a medicament.

13. The conjugate of claim 11 for use in the treatment of a neurodegenerative disease, in particular Gaucher's disease and Parkinson's disease.
